(19) United States
(12) Patent Application Publication
(10) Pub. No.: US 2024/0398740 A1
(43) Pub. Date: Dec. 5, 2024

(54) THERAPEUTIC COMPOSITION, METHODS, AND USES FOR THE CONTROL OF SEIZURES

(71) Applicant: NEUROPRO THERAPEUTICS, INC., Yachats, OR (US)

(72) Inventor: Daryl W. HOCHMAN, Yachats, OR (US)

(21) Appl. No.: 18/680,463

(22) Filed: May 31, 2024

Related U.S. Application Data
(63) Continuation of application No. PCT/US2023/030238, filed on Aug. 15, 2023.
(60) Provisional application No. 63/398,480, filed on Aug. 16, 2022.

(51) Int. Cl. A61K 31/196; A61P 25/08

(52) U.S. Cl. CPC A61K 31/196; A61P 25/08

(57) ABSTRACT

Described herein are compositions that comprise Bumetanide Dibenzylamide for treating selected conditions of the central and peripheral nervous systems employing non-synaptic mechanisms. More specifically, the present disclosure relates to methods and compositions for treating neurological disorders by administering agents that disrupt hypersynchronized neuronal activity without diminishing neuronal excitability. These compositions are useful for seizure disorders, epilepsy, and related indications.

### CROSS-REFERENCE TO RELATED APPLICATIONS

The present application is a continuation application of International Patent Application No. PCT/US2023/030238, filed Aug. 15, 2023, the entire contents of which are hereby incorporated by reference in its entirety. International Patent Application No. PCT/US2023/30238 claims benefit to U.S. Provisional Application Ser. No. 63/398,480, filed Aug. 16, 2022, which is incorporated herein by reference in its entirety.

### TECHNICAL FIELD

Described herein are compositions that comprise Bumetanide Dibenzylamide for treating selected conditions of the central and peripheral nervous systems employing non-synaptic mechanisms. More specifically, the present disclosure relates to methods and compositions for treating neurological disorders by administering agents that disrupt hypersynchronized neuronal activity without diminishing neuronal excitability. These compositions are useful for seizure disorders, epilepsy, and related indications.

### BACKGROUND

Epilepsy is characterized by abnormal discharges of cerebral neurons and is typically manifested as various types of seizures. Many anti-convulsants originally developed for the treatment of epilepsy and other seizure disorders have also found application in the treatment of non-epileptic conditions, including neuropathic pain, mood disorders (such as bipolar affective disorder), and schizophrenia (for a review of the use of anti-epileptic drugs in the treatment of non-epileptic conditions, see Rogawski and Loscher, Nat. Medicine, 10:685-692, 2004). It has thus been suggested that epilepsy, neuropathic pain, and affective disorders have a common pathophysiological mechanism (Rogawski & Loscher, ibid; Ruscheweyh & Sandkuhler, Pain 105:327-338, 2003), namely a pathological increase in neuronal excitability, with a corresponding inappropriately high frequency of spontaneous firing of neurons. However, only some, and not all, antiepileptic drugs are effective in treating neuropathic pain, and furthermore such antiepileptic drugs are only effective in certain subsets of patients with neuropathic pain (McCleane, Expert. Opin. Pharmacother. 5:1299-1312, 2004).

Epileptiform activity is identified with spontaneously occurring synchronized discharges of neuronal populations that can be measured using electrophysiological techniques. This synchronized activity, which distinguishes epileptiform from non-epileptiform activity, is referred to as “hypersynchronization” because it describes the state in which individual neurons become increasingly likely to dis-charge in a time-locked manner with one another. Hypersynchronized activity is typically induced in experimental models of epilepsy either by increasing excitatory or by decreasing inhibitory synaptic currents. It was therefore assumed that hyperexcitability per se was the defining feature involved in the generation and maintenance of epileptiform activity. Similarly, neuropathic pain was believed to involve conversion of neurons involved in pain transmission from a state of normal sensitivity to one of hypersensitivity (Costigan & Woolf, Jnl. Pain 1:35-44, 2000). The focus on developing treatments for both epilepsy and neuropathic pain has thus been on suppressing neuronal hyperexcitability by either: (a) suppressing action potential generation; (b) increasing inhibitory synaptic transmission, or (c) decreasing excitatory synaptic transmission.

Most agents currently used for treatment target synaptic activity in excitatory pathways by, for example, modulating the release or activity of excitatory neurotransmitters, potentiating inhibitory pathways, blocking ion channels involved in impulse generation, and/or acting as membrane stabilizers. Conventional agents and therapeutic approaches for the treatment of epilepsy and neuropsychiatric disorders thus reduce neuronal excitability and inhibit synaptic firing. One serious drawback of these therapies is that they are nonselective and exert their actions on both normal and abnormal neuronal populations. This leads to negative and unintended side effects, which may affect normal CNS functions, such as cognition, learning and memory, and produce adverse physiological and psychological effects in the treated patient. Common side effects include over-sedation, dizziness, loss of memory and liver damage. However, it has been shown that hypersychronous epileptiform activity can be dissociated from hyperexcitability and that the cation chloride cotransport inhibitor furosemide can reversibly block synchronized discharges without reducing hyperexcited synaptic responses (Hochman et al. Science 270:99-102, 1995).

The cation-chloride co-transporters (CCCs) are important regulators of neuronal chloride concentration that are believed to influence cell-to-cell communication, and various aspects of neuronal development, plasticity, and trauma. The CCC gene family consists of three broad groups: Na⁺—Cl⁻ co-transporters (NCCs), K⁺—Cl⁻ co-transporters (KCCs) and Na⁺—K⁺-2Cl⁻ co-transporters (NKCCs). Na—K—Cl co-transport in all cell and tissues is inhibited by loop diuretics, including furosemide, bumetanide, and benzmetanide. Espinosa et al. and Ahmad et al. have previously suggested that furosemide might be useful in the treatment of certain types of epilepsy (Medicina Espanola 61:280-281, 1969; and Brit. J. Clin. Pharmacol. 3:621-625, 1976). Bumetanide is potentially a more potent drug for treating epilepsy, but it also has a more pronounced diuretic effect. There is therefore a continuing need for methods and compositions for treating neuronal disorders that are not diuretic and that disrupt hypersynchronized neuronal activity without diminishing the neuronal excitability and spontaneous synchronization required for normal functioning of the peripheral and central nervous systems.

### SUMMARY

In one embodiment of the present disclosure Bumetanide Dibenzylamide demonstrated antiseizure activity in a nonhuman primate model, and NKCC inhibition measure by a similar response to bumetanide in a rat anxiolytic bioassay. By contrast, although bumetanide demonstrated a potent diuretic effect, in the primate model, treatment with Bumetanide Dibenzylamide did not increase urine output. Comparing the pharmacologic activity of bumetanide and Bumetanide Dibenzylamide highlights the novel properties of Bumetanide Dibenzylamide as a potential antiseizure therapeutic agent that is not limited by diuretic effects. Taken together, the data of the present disclosure along with the published studies describing the antiseizure effects of bumetanide and furosemide, support the novel and unexpected properties of Bumetanide Dibenzylamide as an adjunct antiseizure therapy. It is believed that the observed antiseizure effects of bumetanide and furosemide are mediated through their antagonism of NKCC1 on neurons and/or glial cells whereas their diuretic effects are mediated through antagonism of renal NKCC2.

One embodiment of the present disclosure includes bumetanide derivatives, and compositions containing such compounds, that demonstrate therapeutically effective seizure blockade with substantially no diuretic effect.

One embodiment of the present disclosure includes bumetanide derivatives, and compositions containing such compounds, that demonstrate therapeutically effective seizure blockade with substantially no diuretic effect, wherein the ratio of seizure-controlling effect to diuretic effect provides a therapeutic effect or therapeutic coefficient. The therapeutic effect is a ratio of a measure of seizure suppression (e.g., frequency of seizure, intensity of seizure etc.) to a measure of diuretic effect of the therapeutic compound (e.g., urine volume, urine ion concentration etc.).

One embodiment of the present disclosure includes a therapeutic effect based on seizure frequency and changes in blood plasma osmolarity (as a measure of dehydration). One embodiment of the present disclosure includes a therapeutic effect based on increase in interspike interval and changes in blood plasma osmolarity (as a measure of dehydration). One embodiment of the present disclosure includes a therapeutic effect based on seizure frequency and change in urine production in a given time period. One embodiment of the present disclosure includes a therapeutic effect based on increase in interspike interval and changes in urine production in a given time period.

One embodiment of the present disclosure includes a therapeutic effect based on reduction in seizure spike height or amplitude and changes in blood plasma osmolarity (as a measure of dehydration). One embodiment of the present disclosure includes a therapeutic effect based on reduction in seizure spike height or amplitude and changes in urine production in a given time period. One embodiment of the present disclosure includes a therapeutic effect based on seizure frequency and changes in blood ions over time, where the ions are selected from sodium, chloride, or magnesium. One embodiment of the present disclosure includes a therapeutic effect based on increase in interspike interval and changes in blood ions over time, where the ions are selected from sodium, chloride magnesium, or pH. One embodiment of the present disclosure includes a therapeutic effect based on reduction in seizure spike height or amplitude and changes in blood ions over time, where the ions are selected from sodium, chloride, or magnesium.

Changes in seizure activity may be changes in the amplitude and/or frequency of pharmacologically- or electrically-evoked seizure (epileptiform) activity as measured with EEG or other electrophysiological types of recordings. Changes in seizure activity may be changes in the number of unprovoked seizures over a period of time (such as seizures per day, per week, or per month).

In one embodiment, the therapeutic effect is defined as a proportional change in seizure frequency or amplitude to urine output compared to a baseline. In one embodiment, the therapeutic effect is proporational change in seizure frequency or amplitude in any objective determination. In one embodiment, the therapeutic effect is proporational change in seizure frequency or amplitude pre and post treatment with the therapeutic compound.

In one embodiment, change in frequency post treatment is at least a 50% reduction in the frequency of seizure occurrence. In one embodiment, the change in diuresis post treatment is less than about a two-fold increase in urine production over a twenty four hour period. In one embodiment, change in frequency post treatment is more than a 50% reduction in the frequency of seizure occurrence. In one embodiment, the change in diuresis post treatment is no increase in urine production over a twenty four hour period.

In one embodiment, change in frequency post treatment is about a 50% to a 100% reduction in the frequency of seizure occurrence. In one embodiment, the change in diuresis post treatment is in the range of about 0% to about 100% increase in urine production over a twenty four hour period.

In one embodiment, the therapeutic effect for a specific dose of the therapeutic compound is defined as follows:

Therapeutic Effect=[seizure activity post-treatment]/[seizure activity pre-treatment]*[diuresis post-treatment]/[diuresis pre-treatment]

The dose of the therapeutic compund may be below the dosage required to completely block seizure activity. If the dosage is above the amount required to completely block seizures, is to determine a therapeutic effect that is different from zero, and acertain the comparative effects of different therapeutic compunds.

One embodiment of the present disclosure includes bumetanide derivatives, and compositions containing such compounds, that provide an unexpectedly improved increase in seizure control when compared to other bumetanide derivatives. In other words, not all derivatives of bumetanide provide this effect. Rather, the amide derivatives appear to provide a unique effect.

One embodiment of the present disclosure includes bumetanide derivatives, and compositions containing such compounds, that provide an unexpectedly improved decrease in diuresis when compared to other bumetanide derivatives.

One embodiment of the present disclosure includes bumetanide derivatives, and compositions containing such compounds, that demonstrate a reduction in both seizure amplitude and frequency.

One embodiment of the present disclosure includes bumetanide derivatives, and compositions containing such compounds, in a method or use for seizure suppression.

One embodiment of the present disclosure includes bumetanide derivatives, and compositions containing such compounds, that demonstrate a positive impact on synchronous activity without a substantial impact on excitability. One aspect includes specific bumetanide derivatives, and compositions containing such compounds, that provide a therapeutic window of effect.

One embodiment of the present disclosure includes one or more bumetanide derivatives, and compositions containing such compounds, that provide seizure suppression, including decreasing amplitude of spikes and interval between spikes. In one aspect, the amplitude is decresed by about 50% to about 99% and the interval is decreased by about 50% to about 99%. In one aspect, the ampltide approaches zero (0) and the interval approachs infinity.

One embodiment of the present disclosure includes bumetanide derivatives, and compositions containing such compounds, administered in a therapeutically effective dose, which is one or more doses required to observe the seizure suppression/therapeutic effect. One aspect of the disclosure includes a determination of the diuretic factor for a proposed anti-seizure treatment and then calculating the corresponsing therapeutic factor. One aspect of the present disclosure includes the unique relationship between dosage of the bumetanide amide derivatives and lack of diuretic effect. The bumetanide amide derivatives offer unexpected benefit as compared to other bumetanide derivatives. Testing demonstrates markedly different effects for different derivatives and different formulations.

One embodiment of the present disclosure includes a method or use of treating a patient refractory to conventional anticonvulsant medication comprising administering Bumetanide Dibenzylamide. The present disclosure includes methods and uses that provide particular utility to epilepsy patients who are not well-controlled by or otherwise refractory to conventional therapies, such as one or more of phenytoin, carbamazepine, valproate, lamotrigine, levetiracetam, ethosuximide, phenobarbital, and topiramate by administering to the patient a pharmaceutical composition comrpsing Bumetanide Dibenzylamide.

One embodiment of the present disclosure includes a pharmaceutical composition comprising bumetanide dibenzylamide, bumetanide diethylamide or bumetanide morpholinoamide, or a salt thereof, wherein the pharmaceutical composition has a therapeutic effect on seizure blockade in a patient.

One aspect includes wherein the therapeutic effect is a ratio of a measure of seizure suppression to a measure of diuretic effect on the patient. One aspect includes wherein the measure of seizure suppression is frequency of seizure. One aspect includes wherein the measure of seizure suppression is intensity of seizure. One aspect includes wherein the measure of seizure suppression is a change in amplitude of pharmacologically- or electrically-evoked seizure (epileptiform) activity as measured with EEG or other electrophysiological types of recordings. One aspect includes wherein the amplitude is decreased by about 50% to about 99% post treatment with the composition. One aspect includes wherein the measure of seizure suppression is a change in frequency of pharmacologically- or electrically-evoked seizure (epileptiform) activity as measured with EEG or other electrophysiological types of recordings. One aspect includes wherein the measure of diuretic effect is urine volume. One aspect includes wherein the measure of diuretic effect is urine ion concentration. One aspect includes wherein the therapeutic effect is based on seizure frequency and changes in blood plasma osmolarity. One aspect includes wherein the therapeutic effect is based on increase in interspike interval. One aspect includes wherein the interspike interval is decreased by about 50% to about 99% One aspect includes wherein the therapeutic effect is based on increase in interspike interval and changes in blood plasma osmolarity. One aspect includes wherein the therapeutic effect is based on seizure frequency and change in urine production in a given time period. One aspect includes wherein the therapeutic effect is based on increase in interspike interval and changes in urine production in a given time period. One aspect includes wherein the therapeutic effect is based on reduction in seizure spike height or amplitude and changes in blood plasma osmolarity. One aspect includes wherein the therapeutic effect is effect based on reduction in seizure spike height or amplitude and changes in urine production in a given time period. One aspect includes wherein the therapeutic effect is effect based on seizure frequency and changes in blood ions over time, where the ions are selected from sodium, chloride magnesium, or pH. One aspect includes wherein the therapeutic effect is effect based on an increase in interspike interval and changes in blood ions over time, where the ions are selected from sodium, chloride magnesium, or pH. One aspect includes wherein the therapeutic effect is effect based on reduction in seizure spike height or amplitude and changes in blood ions over time, where the ions are selected from sodium, chloride, or magnesium. One aspect includes wherein the therapeutic effect is a proportional change in seizure frequency or amplitude to urine output compared to a baseline. One aspect includes wherein the therapeutic effect is a proportional change in seizure frequency or amplitude in any objective determination. One aspect includes wherein the therapeutic effect is a proportional change in seizure frequency or amplitude pre and post treatment with the composition. One aspect includes wherein the therapeutic effect is a proportional change in seizure frequency and amplitude pre and post treatment with the composition. One aspect includes change is seizure frequency post treatment with the composition is at least a 50% reduction in frequency of seizure occurrence. One aspect includes wherein change in seizure frequency post-treatment with the composition is a more than a 50% to a 100% reduction in the frequency of seizure occurrence. One aspect includes wherein the measure of diuretic effect is a less than about two-fold increase in urine production over a twenty-four hour period post treatment with the composition. One aspect includes wherein the measure of diuretic effect is no increase in urine production over a twenty-four hour period post treatment with the composition. One aspect includes wherein the measure of diuretic effect is about a 0% to about a 100% increase in urine production over a twenty-four hour period post treatment with the composition. One aspect includes wherein the therapeutic effect is determined based on an effective dose of the composition. One aspect includes wherein the therapeutic effect is determined as:

Therapeutic Effect=[seizure activity post-treatment]/[seizure activity pre-treatment]*[diuresis post-treatment]/[diuresis pre-treatment]

One aspect includes wherein the effective dose of the composition is a dosage required to completely block seizure activity. One aspect includes wherein the effective dose of the composition is above the dosage required to completely block seizures. One aspect includes wherein the effective dose of the composition is a dose that causes seizure suppression without causing the diuretic effect. One aspect includes wherein the composition has a positive impact on neuron synchronous activity without a substantial impact on neuron excitability. One aspect includes wherein the composition provides a therapeutic window of effect. One aspect is a composition comprising Bumetanide dibenzylamide. One aspect is a composition comprising Bumetanide morpholinoamide.

One embodiment of the present disclosure includes a method for treating seizures in a patient comprising: administering the pharmaceutical composition of the present disclosure; and reducing seizure activity in the patient without increasing urine output of the patient.

In one aspect, the pharmaceutical composition is administered orally. In one aspect, the pharmaceutical composition is administered once. In one aspect, the pharmaceutical composition is administered once a day for a fixed number of consecutive days. In one aspect, wherein the antiseizure effects of the pharmaceutical composition is mediated through its antagonism of NKCC1 on neurons and/or glial cells. In one aspect, the diuretic effects of the pharmaceutical composition is mediated through its antagonism of renal NKCC2. In one aspect, the pharmaceutical composition is administered once a day for a fixed number of consecutive days. In one aspect, the pharmaceutical composition is administered to treat epilepsy. In one aspect, the pharmaceutical composition is administered in combination with a conventional therapy to treat seizure. In one aspect, urine output is measured by blood ion concentration imbalance. In one aspect, urine output is measured by the magnitude of diuretic effect calculated as amount of bumetanide in blood (concentration) compared to bumetanide dibenzylamide. In one aspect, time to effect as measured by reduction of seizure frequency for bumetanide dibenzylamide is faster than a traditional anti-epileptic. In one aspect, the reduction in seizure frequency is measured by one or more of: a time increment selected from one or more of hours, days, weeks, and months; a seizure diary and a reduction in logged seizure activity; an increase in one or more of interictal (between) and postictal (after) spiking; and diminishing interictal activity as measured by EEG.

One or more embodiments or aspects may be incorporated in a different embodiment or aspect although not specifically described. That is, all embodiments and aspects can be combined in any way to form another embodiment.

### BRIEF DESCRIPTION OF THE DRAWINGS

FIG. 1 illustrates the results of using furosemide in a seizure model.

FIG. 2 illustrates the impact of furosemide on After Discharge (AD) thresholds.

FIG. 3 illustrates the results of using Keppra® in a seizure model.

FIG. 4 illustrates placement of electrodes in a primate seizure model.

FIG. 5 illustrates representative EEG and AD activity.

FIG. 6 illustrates AD activity, duration, average spike height measurement, and area of envelope.

FIG. 7 illustrates the impact of bicuculine spiking.

FIG. 8 illustrates the comparative effect of NPT 2024 and bumetanide on interspike intervals.

FIG. 9 illustrates the comparative effect of NPT 2024 and bumetanide on spikes per minute.

FIG. 10 illustrates the comparative effect of NPT 2024 and bumetanide on average spike height.

FIG. 11 illustrates the comparative effect of NPT 2024 and bumetanide on urine production over time.

FIG. 12 illustrates stability of spiking from acute bicuculline focus, recorded with surface EEG electrodes.

FIG. 13 illustrates comparative effects of Keppra® and bumetanide in the bicuculline spiking NHP seizure model; IV administration.

FIG. 14 illustrates computer identified tops and bottoms of bicuculline-generated spikes

FIG. 15 illustrates a representative figure for measurement of spike height in the bicuculline focus model.

FIG. 16 illustrates EEG recordings from bicuculline focus after IV bumetanide and bumetanide dibenzylamide (NPT 2042) administration: pretreatment, post-treatment, and recovery.

FIG. 17 illustrates oral bumetanide and NPT 2042: surface EEG recordings from bicuculline focus.

FIG. 18 illustrates Change in urine volume production over time in macaque monkeys after oral administration of bumetanide and NPT 2042.

FIG. 19 illustrates the fear-potentiated startle model.

FIG. 20 illustrates rat fear-potentiated startle data: units of startle reflex amplitude per units of voltage.

FIG. 21 illustrates the percentage change in urine production over time to show the comparative effect of bumetanide, bumetanide morpholinoamide, bumetanide diethylamine, and bumetanide dibenzylamide on diuresis.

FIG. 22 illustrates the rate of urine production to show the comparative effect of bumetanide, bumetanide morpholinoamide, bumetanide diethylamine, and bumetanide dibenzylamide on diuresis (urine rate mL/min).

FIG. 23 illustrates the average urine production rate after treatement to show the comparative effect of bumetanide, bumetanide morpholinoamide, bumetanide diethylamine, and bumetanide dibenzylamide on diuresis.

FIG. 24 illustrates the average pretreatment urine rate (mL/min) compared to the maximum post-treatment urine rate (mL/min) after treatment with bumetanide methyl ester.

FIG. 25 illustrates the average pretreatment urine rate (mL/min) compared to the maximum post-treatment urine rate (mL/min) after treatment with bumetanide cyanomethyl ester.

FIG. 26 illustrates the average pretreatment urine rate (mL/min) compared to the maximum post-treatment urine rate (mL/min) after treatment with bumetanide N.N-diethylglycolamide ester.

FIG. 27 illustrates the average pretreatment urine rate (mL/min) compared to the maximum post-treatment urine rate (mL/min) after treatment with bumetanide benzyl ester.

FIG. 28 illustrates the effect of bumetanide dibenzylamide (NPT 2042) dosing on blood urea nitrogen in humans.

FIG. 29 illustrates the effect of bumetanide dibenzylamide (NPT 2042) dosing on creatinine in humans.

FIG. 30 illustrates the effect of bumetanide dibenzylamide (NPT 2042) dosing on serum chloride in humans.

FIG. 31 illustrates the effect of bumetanide dibenzylamide (NPT 2042) dosing on urine specific gravity in humans.

### DETAILED DESCRIPTION

### Definitions

The terms “active ingredient”, “active pharmaceutical ingredient,” and “API” as used herein refer to a pharmaceutical agent, active ingredient, compound, or substance, compositions, or mixtures thereof, that provide a pharmacological, often beneficial, effect.

The term “dose” as used herein denotes any form of the active ingredient formulation that contains an amount sufficient to produce a therapeutic effect with a single administration.

The term “dosage” as used herein refers to the administering of a specific amount, number, and frequency of doses over a specified period-of-time, typically one (1) day.

The terms “active pharmaceutical ingredient load” or “drug load” as used herein refers to the quantity (mass) of the active pharmaceutical ingredient comprised in a single soft capsule fill.

The terms “formulation” or “pharmaceutical composition” or “composition” as used herein refers to the drug in combination with pharmaceutically acceptable excipients.

The term mean “particle size distribution” (PSD) as used herein refers to the mean particle size from a statistical distribution of a range of particle sizes as described herein. The distribution may be a Gaussian, normal distribution, or a non-normal distribution.

The terms such as “d90,” “d50,” and “d10” refer to the percentage (e.g., 90%, 50%, or 10%, respectively) of particle sizes that are less than a specified size, range, or distribution. For example, “d90≤100 μm” as means that 90% of the particle sizes within a distribution of particles are less than or equal to 100 μm.

As used herein, the term “patient” refers to any subject including mammals and humans. The patient may have a disease or suspected of having a disease and as such is being treated with a drug. In some instances, the patient is a mammal, such as a human, non-human primate, dog, cat, horse, cow, goat, pig, rabbit, rat, mouse, or a premature neonate, neonate, infant, juvenile, adolescent, or adult thereof. In some instances, the term “patient,” as used herein, refers to a human (e.g., a man, a woman, or a child). In some instances, the term “patient,” as used herein, refers to laboratory animal of an animal model study. The patient or subject may be of any age, sex, or combination thereof.

The terms “biological sample” or “sample” as used herein refers to a sample obtained or derived from a patient. By way of example, a biological sample comprises a material selected from the group consisting of body fluids, blood, whole blood, plasma, serum, mucus secretions, saliva, cerebrospinal fluid (CSF), bronchoalveolar lavage fluid (BALF), urine, fluids of the eye (e.g., vitreous fluid, aqueous humor), lymph fluid, lymph node tissue, spleen tissue, bone marrow, and fluid from the auditory cavity.

The term “treating” refers to administering a therapy in an amount, manner, or mode effective (e.g., a therapeutic effect) to improve a condition, symptom, disorder, or parameter associated with a disorder, or a likelihood thereof.

The following abbreviations associated with the description of pharmacology may be used:

| | |
|---|---|
| CNS | central nervous system |
| DMSO | dimethyl sulfoxide |
| ECG | electrocardiogram, electrocardiography |
| HEK | human embryonic kidney |
| hERG | human ether-à-go-go-related gene |
| IC₅₀ | half-maximal inhibitory concentration |
| kg | kilogram |
| μM | micromolar |
| mg | milligram |
| SEDDS | self-emulsifying drug delivery systems |
| SEM | standard error of the mean |

The term “prophylaxis” refers to preventing or reducing the progression of a disorder, either to a statistically significant degree or to a degree detectable to one skilled in the art.

The terms “essentially” or “substantially” as used herein mean to a great or significant extent, but not completely.

The term “about” as used herein refers to any values, including both integers and fractional components that are within a variation of up to ±10% of the value modified by the term “about.”

Also described herein are pharmaceutical compositions and dosage forms comprising one or more agents that reduce the rate by which the compositions described herein as active ingredients will decompose. Such agents, which are referred to herein as “stabilizers,” include, but are not limited to, antioxidants such as ascorbic acid, pH buffers, salts, sugars, etc.

The phrase ‘solubilizer’ is used to refer to an ingredient or group of ingredients that helps solubilize the composition or part of the composition.

The phrases and terms “can be administered by injection”, “injectable”, or “injectability” refer to a combination of factors such as a certain force applied to a plunger of a syringe containing the formulations described herein and at a certain temperature, a needle of a given inner diameter connected to the outlet of such syringe, and the time required to extrude a certain volume of the Bumetanide Dibenzylamide composition from the syringe through the needle.

The range for each ingredient in the described formulation represent the space in which a suitable alternative(s) may be obtained in combination with the other ingredients in ratios adjusted to total 100% w/w. The ranges provided are estimates based on the available data.

### Pharmaceutical Compositions of the Present Disclosure

One embodiment described herein, is a pharmaceutical composition comprising Bumetanide Dibenzylamide. In one aspect, the composition comprises any of the formulations shown in the Tables or Examples described herein. Any of the components in the formulations described herein, shown in the Tables, or illustrated in the Examples can be increased, decreased, combined, substituted, or omitted to provide for a formulation comprising about 100% by weight. Such compositions are hereby disclosed as if they were expressly disclosed herein.

One embodiment described herein is a pharmaceutical composition comprising Bumetanide Dibenzylamide, and one or more solubilizers. Another embodiment described herein is a pharmaceutical composition comprising Bumetanide Dibenzylamide. Another embodiment described herein is a pharmaceutical composition further comprising one or more additional solvents. Another embodiment described herein is a pharmaceutical composition further comprising one or more surfactants, co-surfactants, emulsifying agent or wetting agent. Another embodiment described herein is a pharmaceutical composition consisting essentially of Bumetanide Dibenzylamide. Another embodiment described herein is a pharmaceutical composition consisting essentially of aqueous Bumetanide Dibenzylamide. Another embodiment described herein is a pharmaceutical composition comprising Bumetanide Dibenzylamide, and one or more solubilizers. Another embodiment described herein is a pharmaceutical composition consisting essentially of Bumetanide Dibenzylamide, and one or more solubilizers. In one aspect, the composition is a dry powder compressed into a tablet. In one aspect, the composition is a dry powder filled into a capsule. In one aspect, the composition is a dry powder extruded into a film. In one aspect, the composition is a dry powder extruded into a tablet. One embodiment described herein is a pharmaceutical composition comprising about 2.5 mg to about 42 mg of bumetanide dibenzylamide.

One embodiment described herein is a pharmaceutical composition formulated as an oral capsule. In one aspect, the composition comprises up to about 0.25% w/w to about 15% w/w of Bumetanide Dibenzylamide, and one or more solubilizers. In one aspect, the solubilizer is a co-solvent. In one aspect, the solubilizer is a surfactant. In one aspect, the solubilizer comprises triglycerides. In one aspect, the triglycerides comprises medium chain triglycerides. In one aspect, the triglycerides comprises long chain triglycerides. In one aspect, the triglycerides comprises a mixture of medium and long chain triglycerides. In one aspect, the triglycerides comprises polyoxylglycerides. In one aspect, the polyoxylglycerides is selected from a group consisting Lauroyl Polyoxylglycerides, Linoleoyl Polyoxylglycerides, Oleoyl Polyoxylglycerides, Stearoyl Polyoxylglycerides, Caprylocaproyl Polyoxylglyceride, and any combination thereof. In one aspect, the triglyceride comprises a non-ionic surfactant, solubilizer, emulsifying agent. In one aspect, the long chain triglycerides are selected from a group consisting of Polyoxyl 35 Castor Oil (Kolliphor EL), Glyceryl Monolinoleate (Maisine CC), and any combination thereof. In one aspect, the medium chain triglycerides are selected from a group consisting of Caprylocaproyl Polyoxylglycerides (Labrasol ALF), Phosphatidylcholine (Phosal 75 SA), Caprylic/Capric Triglyceride (Captex 300), Lauroyl Plyoxyl-32 Glycerides (Gelucire 44/14), Sorbitan Ester (Span 80), and any combination thereof.

In one aspect, described herein is a pharmaceutical composition comprising about 0.1% w/w to about 99.75% w/w of one or more solubilizers. In one aspect, the solubilizer comprises soybean oil. In one aspect, the solubilizer is in the oil phase. In one aspect, the solubilizer is selected from a group consisting of Arachis oil, soybean oil, castor oil, corn oil, safflower oil, olive oil, apricot kernel oil, sesame oil, cotton-seed oil, sunflower seed oil, palm oil and rapeseed oil, Maisine 35-1, Maisine CC (Glyceryl Monolinoleate), and any combination thereof. In one aspect, the solubilizer comprises a co-solvent. In one aspect, the solubilizer is selected from a group consisting of Propylene Glycol, Capryol™ 90 (Propylene glycol monocaprylate), Lauroglycol™ 90 (Propylene glycol monolaurate), Glycerin, Polyethylene Glycol, and any combination thereof. In one aspect, the solubilizer comprises an antioxident. In one aspect, the solubilizer is selected from a group consisting of alpha tocopherol, ascorbyl palmitate, ascorbic acid, butylated hydroxyanisole, butylated hydroxyltoluene and any combination thereof. In one aspect, the solubilizer comprises an antimicrobial preservative, a solvent, and a water-soluble co-solvent. In one aspect, the solubilizer comprises a solvent, and a water-soluble co-solvent. In one aspect, the solubilizer is selected from a group consisting Ethanol, Propylene Glycol, Propylene Glycol 300, Propylene Glycol 400, Propylene Glycol 600, Oleyl Alcohol, and any combination thereof. In one aspect, the solubilizer is water. In one aspect, the solubilizer is any diluent.

In one aspect, described herein is a pharmaceutical composition comprising about 0.5% w/w to about 1.8% w/w of Bumetanide Dibenzylamide. In one aspect, described herein is a pharmaceutical composition comprising about 9 mg of Bumetanide Dibenzylamide per capsule to about 12 mg of Bumetanide Dibenzylamide per capsule. In one aspect, described herein is a pharmaceutical composition comprising about 0% w/w to about 1.8% w/w of Bumetanide Dibenzylamide, about 10% w/w to about 45% w/w of Polyoxyl 35 Castor Oil (Kolliphor EL), about 15% w/w to about 65% w/w of Glyceryl Monolinoleate (Maisine CC), about 15% w/w to about 65% w/w of Soybean Oil, about 0% w/w to about 15% w/w of Ethanol, and about 0% w/w to about 0.13% w/w of Butylated Hydroxytoluene. In one aspect, described herein is a pharmaceutical composition comprising about 1.75% w/w of Bumetanide Dibenzylamide, about 32.37% w/w of Polyoxyl 35 Castor Oil (Kolliphor EL), about 31.30% w/w of Glyceryl Monolinoleate (Maisine CC), about 31.30% w/w of Soybean Oil, about 3.25% w/w of Ethanol, and about 0.3% w/w of Butylated Hydroxytoluene.

One embodiment described herein is a pharmaceutical composition formulated as a nasal solution. In one aspect, the composition comprises Bumetanide Dibenzylamide in a solvent system. In one aspect, the composition comprises about 3 ml of solvent and from about 28 mg of Bumetanide Dibenzylamide to about 32 mg of Bumetanide Dibenzylamide. In one aspect, the solubilizer comprises triglycerides. In one aspect, the triglycerides comprises medium chain triglycerides. In one aspect, the triglycerides comprises long chain triglycerides. In one aspect, the triglycerides comprises a mixture of medium and long chain triglycerides. In one aspect, the triglycerides comprises polyoxylglycerides. In one aspect, the polyoxylglycerides is selected from a group consisting Lauroyl Polyoxylglycerides, Linolcoyl Polyoxylglycerides, Oleoyl Polyoxylglycerides, Stearoyl Polyoxylglycerides, Caprylocaproyl Polyoxylglyceride, and any combination thereof.

In one aspect, the triglyceride comprises a non-ionic surfactant, solubilizer, emulsifying agent. In one aspect, the solubilizer comprises Caprylocaproyl Polyoxylglycerides (Labrasol ALF). In one aspect, the solvent system comprises one or more solubilizers. In one aspect, the solubilizer comprises Caprylocaproyl Polyoxylglycerides (Labrasol ALF), and water. In one aspect, the solubilizer comprises Caprylocaproyl Polyoxylglycerides (Labrasol ALF), Propylene Glycol, and water. In one aspect, the solubilizer comprises Caprylocaproyl Polyoxylglycerides (Labrasol ALF), Propylene Glycol, PEG-400, and water. In one aspect, the solubilizer comprises Caprylocaproyl Polyoxylglycerides (Labrasol ALF), Propylene Glycol, PEG-400, Vitamin E TPGS, and water. In one aspect, the solubilizer comprises Caprylocaproyl Polyoxylglycerides (Labrasol ALF), Propylene Glycol, PEG-400, Vitamin E TPGS, Ethanol, and water. In one aspect, the solubilizer comprises about 50 g of Caprylocaproyl Polyoxylglycerides (Labrasol ALF), per 50 g of solvent. In one aspect, the solubilizer comprises about 25 g of Caprylocaproyl Polyoxylglycerides (Labrasol ALF), and about 25 g of water per 50 g of solvent. In one aspect, the solubilizer comprises about 6 g of Propylene Glycol, about 40 g of PEG-400, and about 4 g of water per 50 g of solvent. In one aspect, the solubilizer comprises about 5 g of Caprylocaproyl Polyoxylglycerides (Labrasol ALF), about 6 g of Propylene Glycol, about 35 g of PEG-400, and about 4 g of water per 50 g of solvent. In one aspect, the solubilizer comprises about 10 g of Propylene Glycol, about 35 g or PEG-400, about 0.5 g of Vitamin E TPGS, and about 4.5 g of water per 50 g of solvent. In one aspect, the solubilizer comprises about 5 g of Caprylocaproyl Polyoxylglycerides (Labrasol ALF), about 10 g of Propylene Glycol, about 28.5 g of PEG-400, about 0.5 g of Vitamin E TPGS, about 1 g of Ethanol, and about 4.5 g of water per 50 g of solubilizer. In one aspect, the solubilizer comprises glycofurol. In one aspect, the solubilizer comprises a penetration agent, and a solvent. In one aspect, the solubilizer comprises ethyl oleate. In one aspect, the solubilizer comprises an oleaginous vehicle, solvent, and a solvent.

In one aspect, the pharmaceutical composition comprises comprises about 3% w/v of Bumetanide Dibenzylamide, about 11% w/w of Caprylocaproyl Polyoxylglycerides (Labrasol ALF), about 13.26% w/v of Propylene Glycol, about 73.94% w/v of PEG-400, and about 8.8% w/w of water. In one aspect, the pharmaceutical composition comprises comprises from about 0.01% w/w to about 40% w/w of Bumetanide Dibenzylamide, from about 5% w/w to about 100% w/w of Caprylocaproyl Polyoxylglycerides (Labrasol ALF), from about 4% w/w to about 20% w/w of Propylene Glycol, from about 50% w/w to about 80% w/w of PEG-400, and from about 0% w/w to about 10% w/w of water. In one aspect, the pharmaceutical composition comprises comprises about 2.73% w/w of Bumetanide Dibenzylamide, about 8% w/w of Caprylocaproyl Polyoxylglycerides (Labrasol ALF), about 15% w/w of Propylene Glycol, about 69.27% w/w of PEG-400, and about 5% w/w of water. In one aspect, the pharmaceutical composition comprises comprises about 2.73% w/w of Bumetanide Dibenzylamide, about 16% w/w of Caprylocaproyl Polyoxylglycerides (Labrasol ALF), about 20% w/w of Propylene Glycol, about 54.27% w/w of PEG-400, and about 7% w/w of water. In one aspect, the pharmaceutical composition comprises comprises about 2.73% w/w of Bumetanide Dibenzylamide, about 5% w/w of Caprylocaproyl Polyoxylglycerides (Labrasol ALF), about 4% w/w of Propylene Glycol, about 78.27% w/w of PEG-400, and about 10% w/w of water.

One embodiment described herein is a pharmaceutical composition formulated as a rectal paste. One embodiment described herein is a composition formulated as a rectal gel. In one aspect, the composition is formulated with with a target of about 6 mg Bumetanide Dibenzylamide per gram of the composition based on a target dose of about 30 mg of Bumetanide Dibenzylamide in an amount of about 5 g of the composition. In one aspect, the composition is formulated with a different target dose of Bumetanide Dibenzylamide. In one aspect, the paste is determined to be a 100% non-aqueous formulation in case the drug substance exhibited some instability in water. In one aspect, the rectal gel is formulated to comprise about 0.6% w/w Bumetanide Dibenzylamide, about 10% w/w Caprylocaproyl Polyoxylglycerides (Labrasol ALF), about 9.15% w/w Propylene Glycol, about 53.84% w/w Polyethylene Glycol 600, about 3.6% w/w Polyvinylpyrrolidone (K30), about 2.4% w/w Poloxamer 407 (P407), about 0.41% w/w Sodium Carboxymethyl Cellulose (CMC), and about 20% w/w water. In one aspect, the rectal paste is formulated to comprise about 0.6% w/w Bumetanide Dibenzylamide, about 10% w/w Caprylocaproyl Polyoxylglycerides (Labrasol ALF), about 9.15% w/w Propylene Glycol, about 66.75% w/w Polyethylene Glycol 600, about 3.6% w/w Polyvinylpyrrolidone (K30), about 2.4% w/w Poloxamer 407 (P407), and about 7.5% w/w Polyethylene Glycol 3350.

One embodiment described herein is a pharmaceutical composition formulated as a sublingual tablet. In one aspect, the composition formulated is targeted to have about 30 mg Bumetanide Dibenzylamide per tablet. In one aspect, a small tablet size is used. In one aspect, wetting and or dissolving of the composition occurs within 30 seconds. In one embodiment, the sublingual tablet is formulated to comprise Bumetanide Dibenzylamide, one or more wetting agent, and one or more super disintegrate. In one aspect, the sublingual tablet is formulated to comprise about 15% w Bumetanide Dibenzylamide, about 20% w Ccolus KG (microcrystalline cellulose), about 51% w Mannogem EZ (spray dried mannitol), about 7% w Polyplasdone XL (super disintegrate), about 3% w Poloxamer 407 (wetting agent), about 1.5% w Citric Acid Monohydrate, about 1% w Cabosil M5P (fumed silica), and about 1.5% w Magnesium Stearate. In one embodiment, the sublingual tablet is formulated to comprise Bumetanide Dibenzylamide, one or more water dispersible surfactant, one or more wetting agent, and one or more super disintegrate. In one aspect, the sublingual tablet is formulated to comprise about 7.4% w Bumetanide Dibenzylamide, about 9.9% w Lauroyl Plyoxyl-32 Glycerides (Gelucire 44/14, a water dispersible surfactant), about 9.9% w Sorbitan Ester (Span 80, a water dispersible surfactant), about 14.8% w Neusilin US2 (Magnesium Aluminometasilicate), about 0.5% w/w Poloxamer 407 (wetting agent), about 0.7% w Citric Acid Monohydrate, about 2% w Cabosil M5P (Fumed Silica), about 54.3% w Polyplasdone XL (Super Disintegrate), and about 0.5% w Magnesium Stearate.

### General Methods of Treatment/Uses/Compounds for Use

One embodiment described herein, the preferred treatment agents and methods of the present disclosure are for use in treating seizures (e.g., partial onset seizures), epilepsy, and/or other indications such as neuropathic pain by modulating or disrupting the synchrony of neuronal population activity in areas of heightened synchronization by reducing the activity of NKCC co-transporters without having a diuretic effect. One embodiment described herein, are preferred treatment agents and methods of the present disclosure for treating seizures that cannot be controlled by existing pharmacotherapeutics such as uncontrolled seizures, intractable seizures, refractory seizures, drug resistant seizures, or medically resistant seizures. One embodiment described herein, are preferred treatment agents and methods of the present disclosure for treating epilepsy syndromes such as Angelman syndrome, benign rolandic epilepsy, CDKL5 disorder, childhood absence epilepsy, Dravet syndrome, GLUT1 deficiency syndrome, hypothalamic hamartoma, infantile spasms (also known as West syndrome), Lennox-Gastaut, PCDH19, progressive myoclonic epilepsy, Rasmussen's encephalitis, ring chromosome 20 syndrome, or reflex epilepsies.

One embodiment described herein, the preferred treatment agents and methods of the present disclosure are for use in treating the epilepsy and/or neurological syndromes that are specific to children sincluding but not limited to Dravett syndrome, infantile spasms, Landau-Kleffner Syndrome, Lennox-Gastaut Syndrome, Rasmussen Syndrome, Benign Rolandic Epilepsy, Benign Occipital Epilepsy, Childhood Absence Epilepsy, Juvenile Myoclonic, Rett Syndrome, Angelman Syndrome, Tuberous Sclerosis, and/or Sturge Weber Syndrome. One embodiment described herein, the preferred treatment agents and methods of the present disclosure are for use in treating epilepsy and/or neurological syndromes that may be observed in adults or children.

One embodiment described herein, are preferred treatment agents and methods of the present disclosure for treating one or more indications listed in the following table:

| Old terms for seizure | New term for seizure [choice] (optional) |
|---|---|
| Absence | (Generalized) absence |
| Absence, atypical | (Generalized) absence, atypical |
| Absence, typical | (Generalized) absence, typical |
| Akinetic | Focal behavior arrest, generalized absence |
| Astatic | [Focal/generalized] atonic |
| Atonic | [Focal/generalized] atonic |
| Aura | Focal aware |
| Clonic | [Focal/generalized] clonic |
| Complex partial | Focal impaired awareness |
| Convulsion | [Focal/generalized] motor [tonic-clonic, tonic, |
| | clonic], focal to bilateral tonic-clonic |
| Dacrystic | Focal [aware or impaired awareness] emotional |
| | (dacrystic) |
| Dialeptic | Focal impaired awareness |
| Drop attack | [Focal/generalized] atonic, |
| | [focal/generalized] tonic |
| Fencer's posture | Focal [aware or impaired awareness] |
| (asymmetric tonic) | motor tonic; |
| Figure-of-4 | Focal [aware or impaired awareness] |
| | motor tonic |
| Freeze | Focal [aware or impaired awareness] behavior |
| | arrest |
| Frontal lobe^{a} | Focal |
| Gelastic | Focal [aware or impaired awareness] emotional |
| | (gelastic) |
| Grand mal | Generalized tonic-clonic, focal to |
| | bilateral tonic-clonic, unknown onset tonic- |
| | clonic |
| Gustatory | Focal [aware or impaired awareness] sensory |
| | (gustatory] |
| Infantile spasms | [Focal/generalized/unknown] |
| | onset epileptic spasms |
| Jacksonian | Focal aware motor (Jacksonian) |
| Limbic | Focal impaired awareness |
| Major motor | Generalized tonic-clonic, focal-onset bilateral |
| | tonic-clonic |
| Minor motor | Focal motor, generalized myoclonic |
| Myoclonic | [Focal/generalized] myoclonic |
| Neocortical^{a} | Focal aware or focal impaired awareness |
| Occipital lobe^{a} | Focal |
| Parietal lobe^{a} | Focal |
| Partial | Focal |
| Petite mal | Absence |
| Psychomotor | Focal impaired awareness |
| Rolandic | Focal aware motor, focal to bilateral |
| | tonic-clonic |
| Salaam | [Focal/generalized/unknown onset] epileptic |
| | spasms |
| Secondarily generalized | Focal to bilateral tonic-clonic |
| Tonic-clonic | |
| Simple partial | Focal aware |
| Supplementary motor | Focal motor tonic |
| Sylvian | Focal motor |
| Temporal lobe^{a} | Focal aware/impaired awareness |
| Tonic | [Focal/generalized] tonic |
| Tonic-clonic | [Generalized/unknown] onset tonic - clonic, |
| | focal to bilateral tonic-clonic |
| Uncinate | Focal [aware impaired awareness] sensory |
| | (olfactory) |

Note that there is not a one-to-one correspondence, reflecting reorganization as well as renaming.
The most important terms are set in bold
^{a}Anatomic classification may still be useful for some purposes, for example, in evaluation for epilepsy surgery.

### Epilepsia, 58 (4): 531-542, 2017

doi: 10.1111/ep.13671

One embodiment described herein, are preferred treatment agents and methods of the present disclosure for treating comorbidities of epilepsy or seizures such as psychiatric disorders such as depression, anxiety disorders, attention deficit hyperactivity disorder (ADHD), schizophrenia-like interictal psychosis, autism, as well as suicidal behavior, sleep disorders, austism spectrum disorders, migraines, postictal headaches, depression, anxiety, psychosis, Attention Deficit Disorder (ADD) and Attention Deficit/Hyperactivity Disorder (ADHD), or mental retardation.

One embodiment described herein, are preferred treatment agents and methods of the present disclosure for treating migraines or tinnitus. In one aspect, the preferred treatment agents and methods of the present disclosure may be used to treat migraines with or without aura in adults. In another aspect, the preferred treatment agents and methods of the present disclosure may be used for acute treatment of migraine with auro, acute treatment of migraine without auro, or for chronic treatment for the prevention of migraine with our without aura.

One embodiment described herein, are preferred treatment agents and methods of the present disclosure for treating mild, moderate, or severe anxiety. In one aspect, the preferred treatment agents and methods of the present disclosure may be used for the acute and maintenance treatment of Major Depressive disorder (MDD) in adults and adolescents aged 12-17 years, or for the acute treatment of Generalized Anxiety Disorder (GAD) in adults. In one aspect, the preferred treatment agents and methods of the present disclosure may be used for the acute and maintenance treatment of Obsessive Compulsive Disorder (OCD), acute and maintenance treatment of Bulimia Nervosa, or acute treatment of Panice Disorder (PD) with or without agoraphobia. In another aspect, the preferred treatment agents and methods of the present disclosure may be used for treating acute depressive episodes associated with bipolar I disorder or for treating treatment resistant depression.

One embodiment described herein, are preferred treatment agents and methods of the present disclosure may be used for treating obsessions and compulsions in patients with Obsessive Compulsive Disorder (OCD), Major Depressive Disorder (MDD), Panic Disorder (PD), Social Anxiety Disorder (SAD), Pre-menstrual dysphoric disorder (PMDD), or Posttraumatic Stress Disorder (PTSD). In one aspect, the obsessions or compulsions may cause marked distress, be time-consuming, or significantly interfere with social or occupational functioning, in order to meet the DSM-III-R (circa 1989) diagnosis of OCD. Obsessions may be recurrent, persistent ideas, thoughts, images, or impulses that are egodystonic. Compulsions may be repetitive, purposeful, and/or intentional behaviors performed in response to an obsession or perfomed in a stereotyped fashion. Compulsions may be recognized by the person as excessive or unreasonable

One embodiment described herein, are preferred treatment agents and methods of the present disclosure for treating psychoneurotic patients with mild, moderate, or severe depression, anxiety associated with depression, anxiety as sociated with alcoholism, depression and/or anxiety as so ciated with organic disease, psychotic depressive disorders with associated anxiety including involutional depression and manic-depressive disorders. In one aspect, the preferred treatment agents and methods of the present disclosure may be used to target symptoms of psychoneurosis such as anxiety, tension, depression, somatic symptoms and concerns, sleep disturbances, guilt, lack of energy, fear, apprehension, and worry.

One embodiment described herein, are preferred treatment agents and methods of the present disclosure for treating depressive illness in patients with depressive neurosis (dysthymic disorder), manic-depressive illness, or with major depressive disorder. In one aspect, the preferred treatment agents and methods of the present disclosure may be used for short-term, long-tern and maintenance treatment of Major Depressive Disorder (MDD), Generalized Anxiety Disorder, Diabetic Peripheral Neuropathic Pain (DPNP), Fibromyalgia (FM) or Chronic Musculoskeletal Pain.

One embodiment described herein, are treatment agents and methods of the present disclosure for treating a progressive neurodegenertative disorder, including, for example, Alzheimer's Disease. In one aspect, the preferred treatment agents and methods of the present disclosure may be used for treatment or cessation of disease progression for one or more of Alzheimer's disease, amyotropihc lateral sclerosis, Friedrich ataxia, Huntington's Disease, Lewy Body disease, Parkinson's disease, or spinal muscular atrophy.

One embodiment described herein, are preferred treatment agents and methods of the present disclosure comprising use as a monotherapy or an adjunctive therapy. In one aspect, the preferred treatment agents and methods of the present disclosure may be used as a monotherapy for adults. In another aspect, the preferred treatment agents and methods of the present disclosure may be used as an adjunctive therapy with additional therapy agents for adults. In another aspect, the preferred treatment agents and methods of the present disclosure may be used as a monotherapy for pediatric patients 2 years of age and older. In another aspect, the preferred treatment agents and methods of the present disclosure may be used as an adjunctive therapy with additional therapy agents for pediatric patients 2 years of age and older. In another aspect, the preferred treatment agents and methods of the present disclosure may be used as a monotherapy for pediatric patients less than 2 years of age. In another aspect, the preferred treatment agents and methods of the present disclosure may be used as an adjunctive therapy with additional therapy agents for pediatric patients less than 2 years of age.

The effective amount of an active pharmaceutical ingredient to be administered therapeutically will depend, for example, upon the therapeutic context and objectives. One having ordinary skill in the art will appreciate that the appropriate dosage levels for treatment will vary depending, in part, upon the concentration of the Bumetanide Dibenzylamide composition, the dosing regimen for which the Bumetanide Dibenzylamide composition is being used, the route of administration, and the subject's size (body weight or body surface area) and condition (the age and general health) of the patient. Accordingly, the dosage may be titrated to obtain the optimal therapeutic effect.

As used herein, bumetanide dibenzylamide includes compositions and formulations containing the bumetanide dibenzylamide, as will be relevant in context.

The frequency of dosing will depend upon the pharmacokinetic parameters of the therapeutic agent incorporated into the Bumetanide Dibenzylamide composition being used. The composition can be administered as a single dose, as two or more doses (which may or may not contain the same amount of the Bumetanide Dibenzylamide) over time, or as a continuous infusion of an injection formulation via an implantation device or catheter. Further refinement of the appropriate dosage is routinely made by those of ordinary skill in the art and is within the ambit of tasks routinely performed by them. A sublingual tablet may also be used for oral administration). Appropriate dosages can be ascertained through use of appropriate dose-response data.

Refinement opportunities may include extended or controlled release oral capsule or tablet or use of a transdermal formulation. Intramuscular data shown below supports the development of a potential transdermal therapy. The intramuscular data shows that Bumetanide Dibenzylamide can be absorbed through the microvessels of the muscle into the circulation, and thus avoiding first-pass metabolism. Hence the Bumetanide Dibenzylamide should also be absorbed by the dermal microvessels, lending itself to a transdermal formulation.

The Bumetanide Dibenzylamide composition can be administered, for example, 1×, 2×, 3×, 4×, 5×, 6×, or even more times per day. One or more doses can be administered, for example, for 1, 2, 3, 4, 5, 6, 7 days, or even longer. One or more doses can be administered, for example, for 1, 2, 3, 4 weeks, or even longer. One or more doses can be administered, for example, for 1, 2, 3, 4, 5, 6, 7, 8, 9, 10, 11, 12 months, 1 year, 2, years, 3 years, 4 years, 5 years, over 5 years, a decade, multiple decades, or even longer. One or more doses can be administered at a regular interval until the subject or subject in need thereof, does not require treatment or prophylaxis of epilepsy. In one aspect, the doses maybe administered orally. In one aspect, the doses maybe administered sublingually. In one aspect, the doses maybe administered intravenously. In one aspect, the doses maybe administered intrarectally. In one aspect, the doses maybe administered intramuscularly. In one aspect, the doses maybe administered intranasally. In one aspect, the doses maybe administered subcutaneously.

In one embodiment, the pharmaceutical composition described herein is administered in one or multiple doses simultaneously. For example, two or more identical doses are administered at one time. In another embodiment, two or more different doses are administered at one time. Such dual or different simultaneous doses can be used to provide an effective amount of the pharmaceutical composition to a subject in need thereof.

In one embodiment, the pharmaceutical compositions described herein may be used to treat, prevent, retard the progression of, delay the onset, ameliorate, reduce the symptoms of, or prophylaxis of epilepsy.

In one embodiment, the Bumetanide Dibenzylamide composition described herein is sufficiently dosed in the composition to provide a therapeutically effective amount in one application. In one aspect, one application of Bumetanide Dibenzylamide composition is sufficient for about 1 day, about 2 days, about 3 days, about 4 days, about 5 days, about 1 week, about 2 weeks, about 3 weeks, about 4 weeks, one month, 2 months, 3 months, 4 months, 6 months, 9 months, one year, 2 years, 3 years, 4 years, or even longer. In one aspect, one application of Bumetanide Dibenzylamide composition is given more than once per day.

Another embodiment, the Bumetanide Dibenzylamide composition described herein is provided as a single dose, meaning that the container in which it is supplied contains one pharmaceutical dose. In another embodiment, the composition is provided as a multiple dose composition, meaning that it contains more than one therapeutic dose. Preferably, a multiple dose composition contains at least 2 doses. Such multiple dose Bumetanide Dibenzylamide composition either can be used for different subjects in need thereof or is intended for use in one subject, wherein the remaining doses are stored after the application of the first dose until needed. In another embodiment, the Bumetanide Dibenzylamide composition is comprised in one or more containers.

Crucial to the efficacy of any treatment by a pharmaceutical composition is the overall systemic bioavailability of the pharmaceutical composition used for said treatment. Surprisingly, lipid formulations may be used to increase the bioavailability and/or lymphatic absorption of a pharmaceutical composition. LogP is one measure of lipophyilicity, and is the octal: water partition coefficient expressed as a log ratio of molecule in octanol relative to water after mixing. The LogP of Bumetanide is about 2.61, the logP of Bumetanide Diethylamide is 3.11, and the logP of Bumetanide Dibenzylamide is about 5.9. A LogP of 3 indicates a 1000-fold greater concentration in octonal than water, so Bumetanide Dibenzyl amide is about 1000—to about 10,000-fold more lipophilic than Bumetanide.

### Detailed Methods of Treatment/Uses/Compounds for Use

As described herein, Bumetanide Dibenzylamide is demonstratated to disrupt the synchrony of neuronal population activity in areas of heightened synchronization. The dosage compositions of Bumetanide Dibenzylamide were developed targeting four (4) routes of administrations in attempt to bypass first-pass metabolism for of the composition and increase overall systemic bioavailability. The routes of administration were chosen based upon their potential to maximize the bioavailability of Bumetanide Dibenzylamide and to produce measurable systemic concentrations of Bumetanide Dibenzylamide. Since Bumetanide Dibenzylamide has been shown to be susceptible to high first-pass metabolism by the liver, the routes of administration were selected to avoid hepatic metabolism.

### Medically Intractable Epilepsy

Seizures that cannot be controlled by existing pharmacotherapeutics are referred to in a number of different ways, including “uncontrolled,” “intractable,” “refractory,” “drug resistant”, or “medically resistant”. It is estimated that 20%-40% of patients with epilepsy (approximately 400,000 Americans) have refractory epilepsy. The total indirect and direct cost of epilepsy in the United States is estimated to be $15.5 billion yearly, with drug resistant patients contributing a major proportion of this cost. In spite of the availability of numerous new drugs to treat epilepsy over the last decade, the efficacy of these new drugs have not proven to be significantly better than older drugs.

Most common AEDs (and their use to treat other neurological and psychiatric disorders in addition to epilepsy). The most commonly prescribed AEDs include valproate (VPA—the most commonly prescribed of all AEDs worldwide.) and its derivative Divalproex Sodium, carbamazepine (Tegretol), phenytoin (Dilantin), the barbiturates (Phenobarbital and Primidone), ethosuximide (Zarontin), clonazepam (Klonopin), lamotrigine (Lamictal), gabapentin (Neurontin), topiramate (Topamax), oxcarbazeipin (Trileptal), and Zonisamide (Zonegran). In addition to their use for treating epilepsy, AEDs are also prescribed to treat numerous other neurological and psychiatric disorders.

Side effects: All currently prescribed antiepileptic drugs (AEDs) are thought to mediate their antiepileptic effects by reducing neuronal or synaptic excitability. Since AEDs affect all neuronal or synaptic targets in the brain indiscriminately, regardless of whether or not they contribute to seizure activity, all AEDs also mediate a spectrum of cognitive, neurological, and psychiatric sided effects. Approximately 25% of patients discontinue their treatment because of intolerable side effects. Treatment failure and poor adherence are very common in patients who experience side-effects from their AEDs. The negative consequences of side-effects can significantly affect the lives of relatives and friends of the patient. Commonly occurring side effects of AEDs include memory problems, fatigue, tremors, gastrointestinal symptoms, osteoporosis, depression, drowsiness, weight gain, nausea, and numerous others. One study in The Netherlands estimated the economic costs of the side effects of epilepsy (in addition to the direct and indirect costs of epilepsy itself) for patients in that country to be $26,675 USD per patient per year.

All commonly used AEDs have some effect on cognition, and these effects can have a considerable impact on epilepsy patients when crucial functions are involved, such as learning in children. The most prevalent of the CNS adverse effects on cognition of CNS drugs are sedation, somnolence, distractibility, insomnia, and dizziness.

Fatigue is a common side effect of most antiepileptic drugs. Fatigue induced by AEDs is a chronic condition that can negatively affect the patients work, social interactions, and family. Stimulants such as amphetamine, dextroamphetamine, and methylphenidate are sometimes used to treat fatigue and daytime somnolence. However, these medications can increase seizure intensity and lower seizure threshold, and hence it isn't desirable to use these drugs to treat fatigue in epilepsy patients.

All antiepileptic drugs are thought to increase the risk of suicidal thoughts or actions. This risk is of sufficient concern that the FDA issued safety alerts on Dec. 15, 2008 and Jan. 31, 2008, and currently requires the labeling of all AEDs to include a warning about an increased risk of suicidal thoughts or actions. This is particularly problematic since epilepsy and other illnesses and psychiatric conditions for which AEDs are prescribed as a treatment (chronic pain, depression, bipolar disorders, and anxiety) all inherently have an increased risk of suicidal behavior. For example, death by suicide in people with epilepsy is more common than the population as a whole (5% vs. 1.4%). Thus, the use of an AED to treat a disorder that already has an associated risk of suicidal behavior, would be expected to further increase that risk.

Every antiepileptic drug studied to date has been shown to have endocrine side effects in both men and women. These can adversely affect fertility, sexuality, thyroid function, bone health. AEDs can alter levels of sex hormones which can cause menstrual disturbances, sexual problems, and reduced fertility. Other side effects that affect physical appearance include weight gain, alopecia (hair loss), acne, and masculine hair distribution in women.

The treatment of epilepsy is impacted by poor patient adherence to existing antiepileptic drugs. As stated above, approximately 25% of patients discontinue their treatment because of intolerable side effects. Up to 50% of all epilepsy patients develop adverse reactions to AEDs, which in turn negatively effects tolerability and adherence. Even if a side effect is not intolerable, if it is unpleasant it can reduce patient adherence to taking their AEDs as prescribed. Non-adherence in epilepsy is estimated to be in the range from 30%-50% [24, 25]. Decreased AED adherence is associated with more than a 3-fold increase in mortality [26]. Periods of nonadherence in patients with epilepsy were also associated with significantly more emergency department visits, hospital admissions, injuries, and fractures.

The treatment of epilepsy is impacted by comorbidities.

General: A recent study determined the prevalence of the most common comorbidities in men and women with epilepsy based on data from commercial health plans. The top 10 comorbidities for women and their relative prevalences were psychiatric diagnosis (16%), hypertension (12%), asthma (11%), hyperlipidemia (11%), headache (7%), diabetes (6%), urinary tract infection (5%), hypothyroidism (5%), anemia (5%), and migraine (4%). For men, the top 10 comorbidities and their relative prevalences were psychiatric diagnosis (15%), hyperlipidemia (12%), hypertension (12%), asthma (8%), diabetes (5%), headache (4%), cancer (4%), coronary artery disease (3%), anemia (3%), and gastroesophageal reflux disease (3%). Seven of the top 10 comorbidities were common to both women and men. Psychiatric diagnosis was the only comorbidity among the top five comorbidities for all age groups. The presence of one comorbidity approximately tripled the health-care cost for that member compared with the cost for members who had no comorbidities.

Psychiatric disorders—general: Epilepsy increases the likelihood of depression, anxiety disorders, attention deficit hyperactivity disorder (ADHD), schizophrenia-like interictal psychosis, autism, as well as suicidal behavior. Likewise, individuals with these psychiatric diagnoses and suicidal behavior are more likely to have epilepsy.

Sleep disorders: Sleep deprivation is known to lower seizure thresholds in people with epilepsy. Sleep is vulnerable to its own set of disorders that can disrupt it. One example is obstructive sleep apnea [OSA]. Both adults and children with refractory epilepsy are at much higher risk than the normal population for developing OSA.

Autism Spectrum Disorder (ASD): Epilepsy occurs at a much higher frequency in individuals with autism. Between 11% and 39% of individuals with autism develop epilepsy [38]. Epilepsy and autism co-exist in up to 20% of children with either disorder. In children with autism, the highest prevalence of epilepsy is in those with intellectual disability.

Sleep disturbances in children with autism are prevalent, with estimates from 40% to 80% of children being affected.

The atypical antipsychotic medications risperidone and aripiprazole are approved by the food and drug administration for the treatment of irritability and agitation in ASD. Both are associated with significant adverse events, including the lowering the seizure threshold.

Migraine: Migraine has an incidence of around 1% per year and a 1-year prevalence of 11.7-13.2% Patients with epilepsy have a roughly twofold increased risk of having migraine as well. Conversely, children with migraine have threefold to fourfold increase in the risk of developing epilepsy.

Comorbidity can worsen outcome. Patients with epilepsy who suffer from migraine are less likely to have a remission of epilepsy than those with epilepsy alone. This is also evidence for a complex comorbid cluster of epilepsy, migraine, depression, and suicide.

Postictal headaches-45% of people with epilepsy have headaches following seizures, called postical headache. These headaches last between 6-24 hours or longer, and can be quite disabling. A number of drugs used to treat these headaches can lower seizure threshold, increasing risk for further seizures.

Depression: Depressive disorders in patients with epilepsy has been shown to range between 9% and 55% depending on the sample population and the methods of assessment. This is in contrast to the prevalence in the general population estimated to be 1%-3% in men, and 2%-9% in women.

The therapeutics commonly used to treat depression can lower seizure thresholds or increase the severity of seizures. Bupropion and tricyclic antidepressants reduce seizure threshold. Selective serotonin reuptake inhibitors (SSRIs) can significantly prolong seizures.

Anxiety: The lifetime prevalence of anxiety is estimated to be 2.4 times higher in people with epilepsy than in people without epilepsy.

Psychosis: The risk of psychosis in patients with epilepsy may be 6-12 times that of the general population, with a prevalence of about 7-8%. All antipsychotic medications can lower seizure thresholds.

Attention Deficit Disorder (ADD) and Attention Deficit/Hyperactivity Disorder (ADHD): Nearly 20% of adults diagnosed with epilepsy also show symptoms of ADHD. Studies in pediatric epilepsy have found a 2.5-fold to 5.5-fold-increased risk of ADHD compared with healthy controls. It is estimated that between 2% and 7% of children with ADHD have epilepsy.

Stimulants such as amphetamine, dextroamphetamine, and methylphenidate are commonly prescribed to treat Attention Deficit Disorder (ADD) and Attention Deficit/Hyperactivity Disorder (ADHD) in children as well as adults. These drugs can reduce seizure threshold and increase seizure severity.

Mental retardation: Epilepsy is one of the most common secondary disabilities in people with mental retardation, the prevalence increasing with the severity of the intellectual disability. About 50% of those with profound learning disability develop epilepsy. The prevalence of lifetime epilepsy among people with mental retardation (IQ<70) is between 13% and 24%. Down Syndrome is the most common genetic cause of mental retardation; the number of people with Down Syndrome who have seizures is estimated to be between 5% and 10%. Currently available AEDs and elicit adverse behavioral effects in individuals with mental retardation.

Others: Hyperlipidemia-incidence rate is 1.3 fold higher in epilepsy patients than in control. Population-based surveys document higher rates of hypertension, ischemic heart disease and diabetes in people with epilepsy.

Drug interactions may create an issue n the treatment of seizure disorders.

The following drugs may lower seizure threshold, thus increasing the risk of seizures in people with epilepsy: Acetylcholinesterase inhibitors—Used to treat: Myasthenia gravis, glaucoma, postural tachycardia syndrome, neuropsychiatric symptoms of Alzheimer's disease, Lewy Body Dementia, Parkinson's disease, cognitive impairments in schizophrenia, autism; Anticholinergics—Used to treat gastrointestinal disorders, genitourinary disorders, respiratory disorders, sinus bradycardia, insomnia, and dizziness; Antiemetics—Used to treat nausea/vomiting; Antihistamines—suppress symptoms of allergic reaction; Baclofen-skeletal muscle relaxant for spasticity; ß-Blockers-Angina pectoris, Atrial fibrillation, Cardiac arrhythmia, Congestive heart failure, Essential tremor, Glaucoma, Hypertension, Migraine prophylaxis, Mitral valve, prolapsed, Myocardial infarction, Phacochromocytoma, in conjunction with α-blocker, Postural orthostatic tachycardia syndrome, Symptomatic control (tachycardia, tremor) in anxiety and hyperthyroidism, Theophylline overdose; Cephalosporins-antibiotics; Cyclosporine—immunosuppressant, used for severe rheumatoid arthritis, severe psoriasis; Dalfampridine—used to treat multiple sclerosis, spinal cord injury, Parkinson's disease; Estrogen—oral contraceptives, hormonal replacement therapy (given postmenopause to prevent osteoporosis, treat the symptoms of menopause, prostate cancer; Imipenem—antibiotic; Iodinated Contrast Dyes—radiocontrast agent; Isoniazid—prevention and treatment of tuberculosis; Lithium—bipolar disorders, major depression, and schizophrenia; Local anesthetics—seizures are a well recognized side effect due to the administration of local anesthetics; Methotrexate—chemotherapy for certain cancers, autoimmune disorders including rheumatoid arthritis, juvenile dermatomyositis, psoriasis, psoriatic arthritis, lupus, sarcoidosis, Crohn's disease, eczema and many forms of vasculitis; Metronidazole-antibiotic and antiprotozoal medication; Narcotics-pain; Penicillins-antibiotics; Pyrimethamine—antimalarial drug, used for protozoal infections; Quinolones—antibacterial drugs; Theophylline—chronic obstructive pulmonary disease (COPD), asthma, infant apnea. Blocks the action of adenosine, an inhibitor neurotransmitter that induces sleep, contracts the smooth muscles and relaxes the cardiac muscle; Tramadol—pain.

Rarer Epilepsy Syndromes may be treated with the compounds and compositions of the present disclosure, including Angelman Syndrome—Occurs in 1/15,000 births. Epilepsy is present in more than 80% of affected individuals; Benign Rolandic Epilepsy—Represents about 15% of all epilepsies in children. Seizures stop by age 15; CDKL5 Disorder—maybe too rare to be of interest. 600 cases worldwide; Childhood Absence Epilepsy—Accounts for 2-8% of people with epilepsy. Usually disappears by adulthood; Dravet Syndrome: Affects 1/30,000. Myclonic seizures appear between 1-5 years in 85% of children; GLUT1 deficiency syndrome—Maybe 1/90,000, but thought to be underdiagnosed because many neurological disorders cause similar symptoms. Almost all individuals have frequent seizures beginning in first year of life; Hypothalamic hamartom— 1/200,000; Infantile spasms (also known as West syndrome)—2.5-6 of every 10,000 births. Accounts for 30% of all cases of epilepsy affecting infants. Usually stops by 4 yrs, but most children left developmentally impaired, and one-fifth will have Lennox-Gastaut syndrome. Many clinicians believe that the sooner the seizures are controlled, but better the outcome; Lennox-Gastaut—accounts for 2-5% of childhood epilepsies. Usually persists through childhood and adolescence to adult years. Seizures very hard to control with current therapeutics; PCDH19—1 in 10 girls that begin giving seizures before age 5 may have PCDH19 epilepsy. Can overlap or look similar to Dravet Syndrome. 15,000-30,000 people in with PCEH19 epilepsy in the United States; Progressive myoclonic epilepsy—Not a single disorder, but includes a group of syndromes with various names, including “Severe myoclonic epilepsy of infancy (Dravet syndrome), Unverrict-Lundbord disease (also called Baltic myoclonus), Lafora disease, and Mitochondrial encephalopathies. Hard to control any of these patients with existing therapeutics; Rasmussen's Encephalitis-Nothing is known about its incidence in different populations. Considered rare, but clinicians all over the world describe patients with this syndrome. Outlook grim with current therapeutics; seizures are relentless; Ring Chromosome 20 Syndrome—rare; Reflex epilepsies—group of epilepsy syndromes in which a certain stimulus (e.g. flickering light) triggers seizures. 4-7% among patients with epilepsy.

Monontherapy vs polytherapy (combination therapy) side effects. Often a single AED might provide partial, but inadequate seizure control. For patients who are refractory to any single AED (monotherapy), better seizure control is sometimes obtained by combining several different AEDs (polytherapy, or combination therapy). Sometimes up to four AEDs are given to a single patient to try to control seizures. However, the intensity and number of side effects significantly increases when more than one AED is given to a patient. A study has showed that polytherapy has more severe and numerous cognitive side effects. The intensity and number of side effects increases with the number of AEDs a patient is taking.

The FDA labeling for bumetanide includes the statement that serum potassium should be measured periodically and potassium supplements or potassium sparing diuretics added if necessary. Periodic determinations of other electrolytes are advised in patients treated with high doses or for prolonged periods, particularly in those on low-salt diets. Hyperuricemia may occur; it has been asymptomatic in cases reported to date. Reversible elevations of the BUN and creatinine may also occur, especially in association with dehydration and particularly in patients with renal insufficiency. Bumex may increase urinary calcium excretion with resultant hypocalcemia. Diuretics have been shown to increase the urinary excretion of magnesium; this may result in hypomagnesemia.

Ideally, it would be desirable to increase the ability of the loop diuretics to pass the blood-brain-barrier. This would have the effect of being able to reach greater therapeutic effects in the brain, and reduced diuretic effects. We have found that certain amide analogs of bumetanide have profound antiepileptic effects, with dramatically reduced diuretic effects by comparison to bumetanide. This is an unexpected discovery. For example, Tollner et al tested a bumetanide amide derivative in their studies (N,N-Dimethylaminoethylamide) in rats, and found that it did not lead to enhanced levels of bumetanide. The authors in this study then chose to abandon further testing of amide derivatives of bumetanide.

The present methods provide particular utility to epilepsy patients who are not well-controlled by or otherwise refractory to conventional therapies, such as, phenytoin, carbamazepine, valproate, lamotrigine, levetiracetam, ethosuximide, phenobarbital, and topiramate.

### A Primate Model

The primate neocortical seizure model was used here for testing the effects of NKCC antagonists on epileptiform activity. This model and the techniques used to analyze the data was originally developed by Haglund and Hochman for the purpose of studying intrinsic optical signals in the human and primate brain, and how these signals could be used to map spread of seizure activity in the neocortex (Haglund et al. 1993, Haglund and Hochman, 2007). In primate studies, epileptiform activity is generated either by electrically stimulating a small focal cortical region with bipolar microelectrodes to generate afterdischarge activity (similar to what is done for intraoperative mapping of seizure foci in human patients; see below), or by generating an acute focus by applying various epileptiform causing agents (e.g. bicuculline, 4-AP) to a focal region over hand motor cortex (similar to recording from an interictal focus in human patients; see below). We found that the data acquired from primate seizure studies were qualitatively similar to what we found when doing similar optical imaging studies in human patients (Haglund and Hochman, 2005), in that the morphology the electrophysiological activity recorded in EEG traces was similar between monkeys and humans. In particular, the spontaneous interictal spiking observed in humans, shown below, is similar to the neocortical spiking generated by a bicuculine focus on the neocortex in the primate model. The afterdischarge protocol developed for human intraoperative studies as shown in the second figure below was also used in primate studies. As well, the physical properties of the optical signals from the brain generated by epileptiform activity (not shown here) (Haglund and Hochman, 2005, Haglund and Hochman 2007).

### Example Comparator—1: Furosemide (NKCC antagonist) Blockade of Spontaneous Interictal Spiking in Human Patients with Medically Intractable Epilepsy (from Haglund and Hochman, 2005)

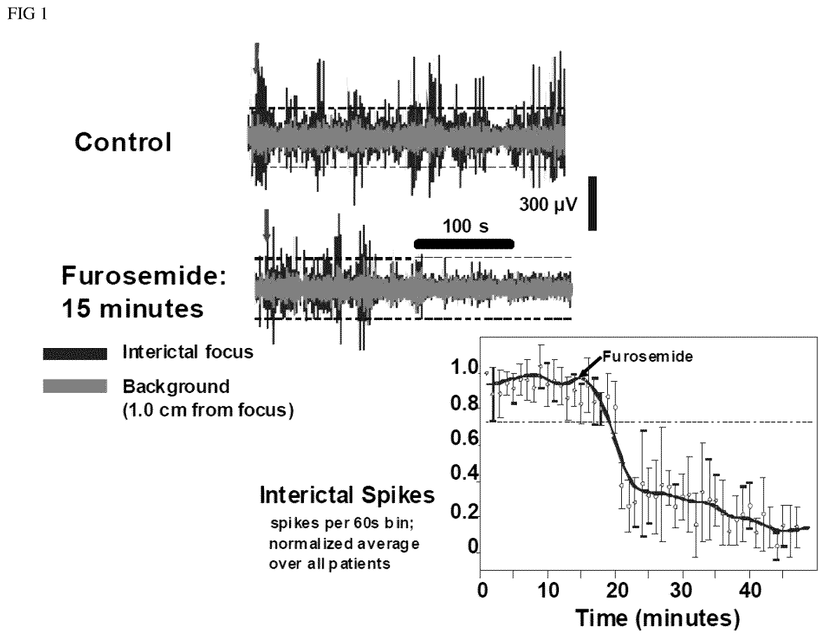

With reference to FIG. 1, the data in this figure were analysed with the methods described herein for the primate studies. Top traces show data from an individual patient to illustrate the changes in spontaneous interictal spiking following furosemide administration. The electrophysiological activity was recorded from EEG electrodes placed on the cortical surface as was done in primates. The Two upper traces compare the activity before and following administration of 20 mg IV furosemide. The dark blue trace was recorded from an electrode at the interictal focus, and the superimposed light blue trace shows background activity from an electrode 1 cm away. The change in spike frequency over time was determined using the same algorithms applied to our primate data, averaged over 5 patients, is shown in the bottom plot.

### Example Comparator—2: Effects of Furosemide (NKCC Antagonist) on Afterdischarge Threshold in the Cortex of Human Subjects (Haglund and Hochman, 2005)

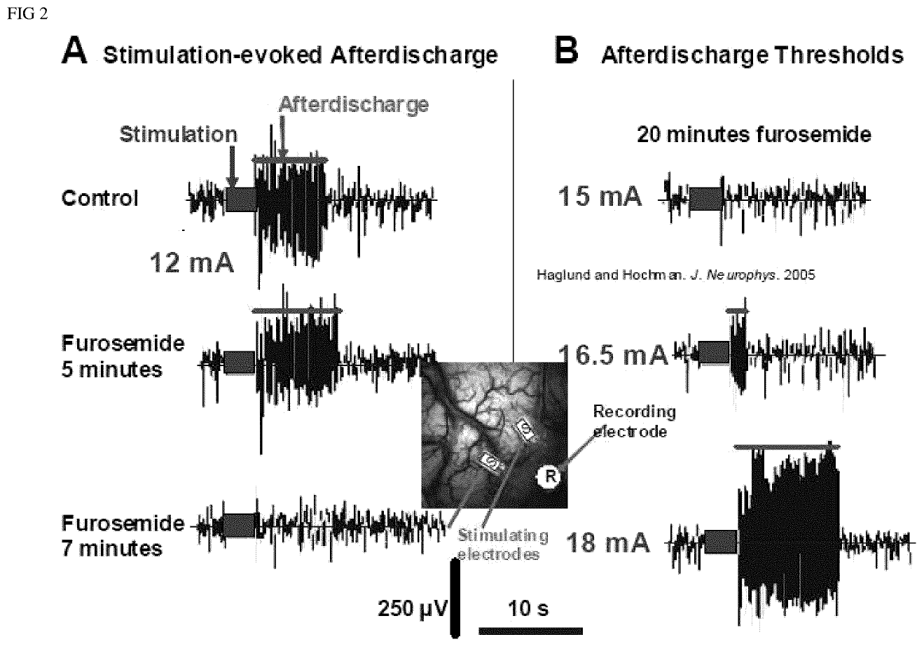

With reference to FIGS. 2, A and B, a bipolar stimulating electrode (similar to the one used in primate studies) was placed on the cortical surface as shown in the gray-scale image in lower center of figure. A recording electrode was placed within 1 cm of the stimulating electrode. A 4-second stimulus (60 Hz; biphasic; 1 ms/phase) was delivered at various currents (similar protocol used in primate studies); the stimulation duration is represented by blue boxes embedded in the beginning of the traces. Prior to furosemide treatment, the minimum current necessary to elicit at least 5 seconds of afterdischarge activity in three consecutive trials was determined; this was defined to be the ‘afterdischarge threshold current’ (A. top). The red horizontal bars above each trace mark the episode of afterdischarge activity. Following furosemide administration, stimulation trials were performed every 2-5 minutes for the next 40 minutes. In this patient, the afterdischarge activity was abruptly blocked soon after furosemide treatment (A. bottom traces). In order to determine whether the blockade of afterdischarge activity was mediated by an increase in afterdischarge threshold, the stimulation current was incrementally increased (B). It was determined that afterdischarge episodes lasting at least as long as those observed during the pre-furosemide trials could be elicited with increased stimulation current. This result suggested that furosemide increased the afterdischarge threshold.

### Example Comparator—3: Quantification of Effects of NKCC2 Antagonists on Diuresis

Rats rapidly biotransform bumetanide into inactive metabolites, and hence bumetanide doesn't elicit diuresis in rats even though it is potently diuretic in humans (Schwartz, 1981. Metabolism of Bumetanide. J Clin PHarmacol. 12:555-563). Similarly, the diuretic effects of bumetanide in dogs is much less than humans because of the rapid renal excretion of bumetanide in dogs (Schwartz, 1981). Studies in Hochman's lab at Duke showed that primates could be catheterized and diuresis quantified. This makes the primate a species that is better suited that other common laboratory species to comparing the diuretic effects of bumetanide analogs to bumetanide in a way that is transferrable to humans.

### Example Comparator—4: Primate Model is Sensitive to Antiepileptic Effects of Drugs that have No Antiepileptic Effects in Rats

Keppra (Levetiracetam) is an excellent antiepileptic drug that faced an uphill battle in its development because it was famously known to not have any antiepileptic effects in the standard rat models of seizures. From the pharmacology review provided to the FDA: “There was no anticonvulsant activity in two screening tests for antiepileptic drugs (AEDs), the maximal electroshock (MES) test and the maximal pentylenetetrazol (PTZ) test. Levetiracetam lacked anticonvulsant action against seizures induced by maximal stimulation with different chemoconvulsants and showed minor anticonvulsant action with submaximal stimulation and also in threshold tests, one exception being the protection observed against seizures induced by pilocarpine and kainic acid.

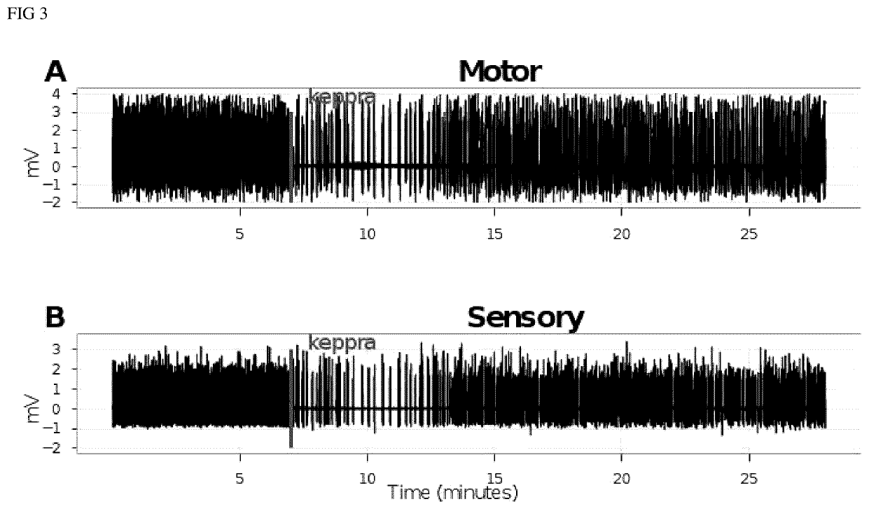

As shown in FIG. 3, a clear anticonvulsant effect of Keppra can be measured in our primate seizure model.

As Keppra did in its time, we believe that bumetanide mediates its antiepileptic effects through mechanisms that are unique to all currently approved AEDs. The primate model seems to show sufficient sensitivity to the anticonvulsant effects of bumenetanide and its analogs so that it can be relied upon for studying these class of molecules in a way that we believe will be transferable to the human.

Furosemide and bumetanide block seizures; their rank-order for antagonizing NKCC is preserved. Furosemide at 10 mg/kg IV, bumetanide at 2 mg/kg IV. Foresemide is about 1/10^{th} as diuretic as bumetanide. Bumetanide, Fosphenatoin (Cerebyx) and pentothal (sodium thiopental) block 4-AP bursting. Keppra 2×40 mg/kg (Levetiracetam) does NOT block 4-AP bursting. Hence primate model is sensitive to Keppra, but Keppra famously was not effective in standard AED rat models. Bumetanide and Keppra block bicuculline spiking, but Fosphenatoin does not. Bumetanide and phosphenatoin block ADs and hand-twitching, Keppra does not.

### EXAMPLES

### Example 1: Synthesis of Bumetanide Dibenzylamide (NPT 2042)

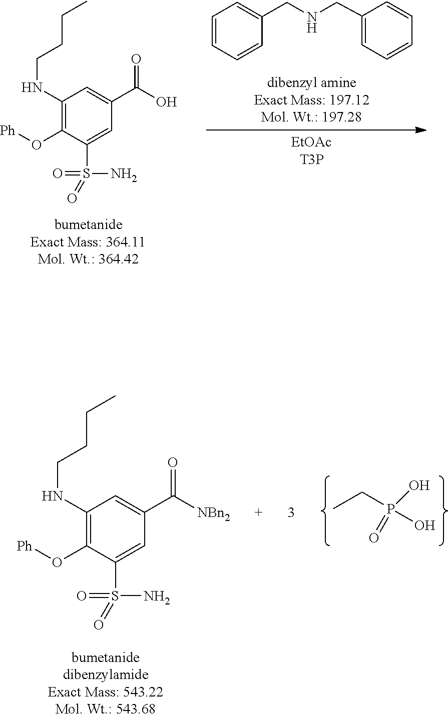

Bumetanide Dibenzlyamide may be made following the procedures set forth in U.S. Pat. No. 8,008,283, herein incorporated by reference in its entirety.

Bumetanide Dibenzylamide may be synthesized as described. Bumetanide (960 mg, 2.6 mmol) was dissolved in dimethyiformamide (DMF, 10 mL) and 1-ethyl-3-(3-dimethy-laminopropyl) carbodiimide (EDC, 560 mg, 3.6 mmol) was added. After about 10 minutes, 1-hydroxybenzotriazole (HOBt, 392 mg, 2.9 mmol) was added and the solution was stirred for an additional 10 minutes. Dibenzylamine (1 mL, 5.2 mmol) was added and the reaction was stirred for 2 hours, at which time the reaction was complete by LC/MS. The reaction was poured into saturated ammonium chloride (20 mL) and extracted with ethyl acetate (2×100 mL). The ethyl acetate, was washed with saturated sodium bicarbonate, water, and brine, and dried over anhydrous magnesium sulfate. The ethyl acetate was removed under reduced pressure to yield 1.0 g (75%) of N, N-dibenzyl 3-aminosulfonyl-5-butylamino-4-phenoxybenzamide (Bumetanide Dibenzylamide) as white solid.

An initial set of amide analogs of bumetanide was first screened by measuring its NKCC-mediated anxiolytic effects in a rat anxiety whole animal bioassay, and comparing those effects to bumetanide, using the same methods as described by Krystal 2012. The rat anxiety model studies were performed by NeuroInvestigations Inc. at the University of Lethbridge (Lethbridge, Canada). Candidates that elicited anxiolytic effects were evaluated for diuresis and antiseizure effects in monkeys (Cynomolgus M. nemistrina) by Drs. Hochman and Haglund at Duke University Medical Center (Durham, NC). The monkeys were anesthetized and catheterized during these experiments, and their urine was collected and measured in a graduated flask so that effects of various treatments on diuresis could be quantified. Cynomolgus M. nemistrina monkeys were selected as the relevant species to study the diuretic effects because it is believed that they metabolize bumetanide (and hence, likely its analogs) similarly to humans (Doyle 1982, Walmsley 1985).

### Example 2: Experimental Set-up and Test Methods

Bumetanide derivatives may be synthesized according to methods as describes in U.S. Pat. No. 8,008,283, previously incorporated by reference, and all tested derivatives were dissolved in PEG 200 for IV formulation. For oral administered in CMC suspension with water and delivered by oral gavage at an equimolar equivalent to 10 mg/kg BUM.

Macaque monkeys (Macaca nemestrina, weight 2-3 kg) were used were used for all the data shown here, with their care and treatment conforming to a protocol approved by Duke's Institutional Animal Care & Use Committee. Details on the treatment and surgical preparation of macaques for cortical AD stimulation and EEG recording have been described (Haglund et al., 1993; Haglund and Hochman, 2007, Tolner, E. A. et al., 2011).

Animals were artificially ventilated on 100% oxygen via intubation (Matrix VBS anesthesia machine and Hallowell EMC model 2002 ventilator) and kept under pentobarbital anesthesia (1-2 mg/kg/hr). Oxygen saturation was measured from the tongue using a tongue-sensor (Nellcor Pulse Oximeter) and was continuously monitored to maintain at constant 98-100% saturation throughout the experiments.

Fluid balance was controlled by monitoring intravenous fluid intake every 15 minutes and fluid output (via a Foley catheter) every 30 minutes. Heart rate, blood pressure (Critikon Dinamap blood pressure monitor, model 8100), end-tidal pCO₂, respiration and O₂ levels (Ohmeda 5250 RGM anesthesia monitor; Nellcor NBP-40 for SpO₂) were monitored in line with the EEG throughout experiments.

After craniectomy (25 mm in diameter), the dura was peeled back and surface EEG recordings were made using custom-built strip electrodes (2.5 mm electrode diameter; 1 cm inter-electrode distance) placed over the sensorimotor cortex, with one electrode overlying the hand motor cortex, one over sensory cortex and a reference electrode attached to the skull at the mastoid process or to a third surface electrode in between the motor and sensory electrodes.

An acute bicuculline focus was generated in the motor cortex by 0.5 mm² pledgets of gelfoam (Codman & Shurtleff, Randolph, MA, USA) soaked in 100 μM bicuculline (Sigma-Aldrich, St. Louis, Missouri, USA). Pledgets were placed on the cortex for five minutes and refreshed for five minutes every hour. The bicuculline-evoked epileptiform spike activity stabilized after about 1 hour, after which data acquisition started. EEG-signals from motor and sensory cortex electrodes were continuously recorded (1000 Hz sampling, no filters; Axon Instrument's Digidata 1440A system).

Stimulation-evoked afterdischarges (Ads) were evoked by a 4 sec train of 60 Hz biphasic pulses at 4-20 mA using a bipolar stimulating electrode (5 mm interelectrode distance) powered by a constant current source (Ojemann Cortical Stimulator, Integra Life Sciences Corporation, NJ, U.S.A.) placed on the sensory cortex, or by passing current between two of the three surface electrodes. The AD threshold was determined by stimulating at the lowest level that reliably triggered AD activity three times in a row and thresholds remained constant throughout recordings. AD activity was reliably elicited on the sensory cortex during the same experiments in which a bicuculline focus was created on the motor cortex. Several AD trials were conducted with the animals with a 20-30 min interval in between. In each AD trial, 3-8 AD stimulations (at 1 minute intervals) were made during the control condition, during onset of the maximum CNS effect from treatment with bumetanide or a bumetanide derivative, and during the recovery phase.

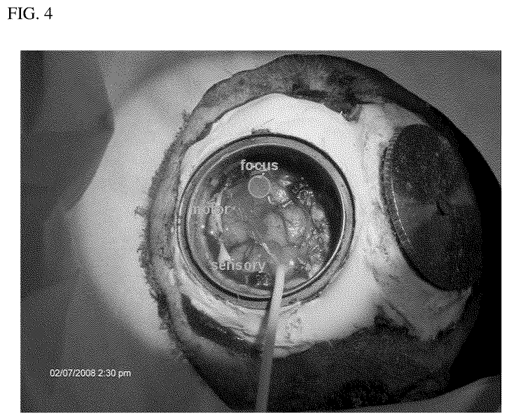

With reference to FIG. 4: Experimental Setup: An acute focus using bicucucline or 4-AP was created near the hand hand motor cortex. Three surface electrodes were place so that one electrode was overlying hand motor cortex, and another overlying hand sensory cortex. The middle electrode was used as a reference for differential recordings. The recordings electrodes could also by used to stimulate the cortex by passing current between a pair of electrodes.

Data were analyzed off-line as described previously (Haglund and Hochman, 2005) with custom-designed software using the R programming language. The bicuculline-induced spike activity was quantified from the motor cortex recording. The effect of treatments on bicuculline-induced spikes was analyzed by comparing the peak response after a treatment was administered to the mean value during the 5 minutes control period before the treatment administration. In the bicuculline experiments, recovery from following pharmacological treatment was followed for a period of 20-35 minutes after return pre-treatment spike-activity. Due to technical reasons, some recordings had to be terminated before the recovery phase.

### Example 3: After Discharge (AD) Activity

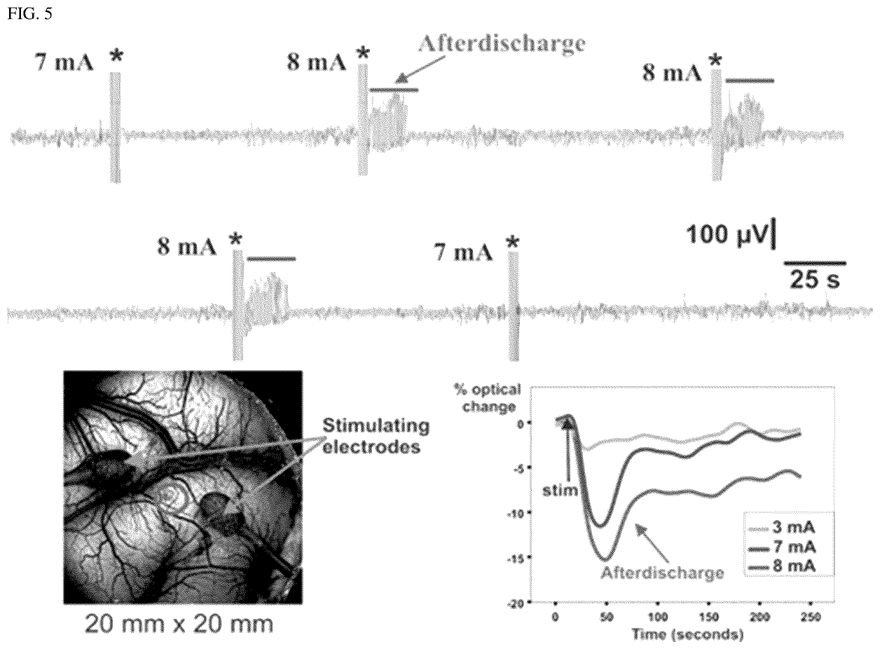

With reference to FIG. 5, the bottom left image shows the position of the stimulating electrodes on the surface of the monkey cortex (in this example, the electrodes of the Ojemann stimulator are being used rather than the surface electrodes shown in FIG. 1). The brain is electrically stimulated for 4 seconds at varying current magnitudes until the “after discharge threshold” is determined: the magnitude of stimulation current that is just sufficient to elicit ADs. The stimulation artifacts are shown in the top lefthand trace, denoted by stars (*). The ADs are shown by orange bars. One can see that, in this case, 7 mA was not sufficient for eliciting ADs, but 8 mA reliably generated ADs. Hence the AD-threshold in this was 8 mA. In this way, changes in the effect of a treatment on this type of electrical stimulation-evoked epileptiform activity can be quantified by measuring changes in the AD-threshold. From Haglund and Hochman, 2007.

### Example 4: Quantification of after Discharge Activity

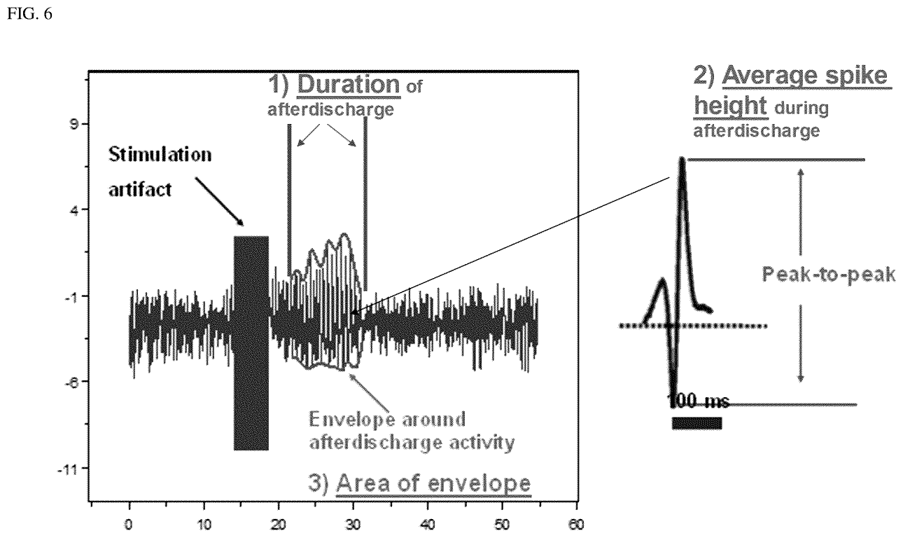

With reference to FIG. 6, this figure shows three aspects of the AD activity, in addition to the AD-threshold described in FIG. 2, that can be quantified using custom software developed by D. Hochman: 1) Durations of the AD activity, 2) average spike-height of the AD activity, and 3) the area within the envelope of the AD activity.

### Example 5: Bicuculline Spike Activity

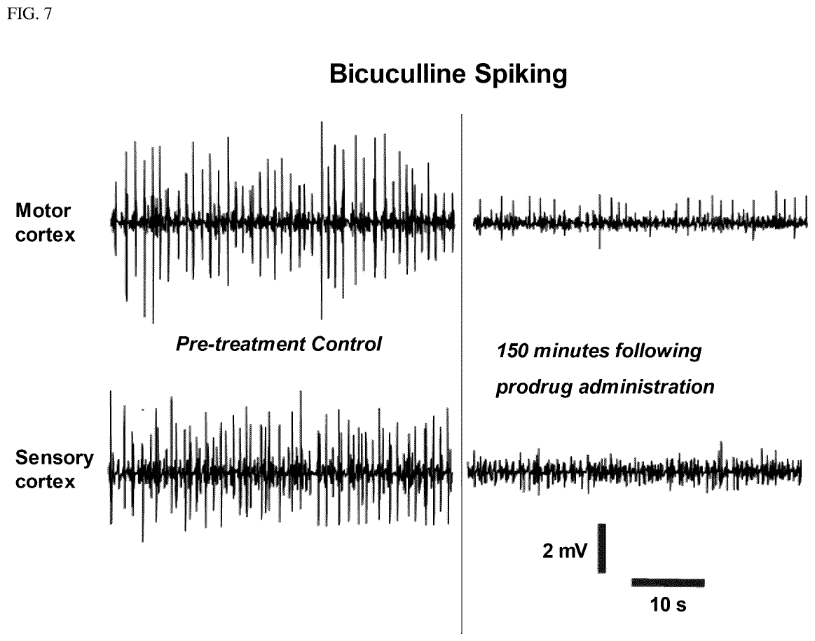

Referring to FIG. 7, this shows the continuous EEG traces recorded from the surface electrodes nearest the epileptic focus in hand motor cortex (upper traces) and nearest hand sensory cortex (lower traces). In addition to visual comparison (i.e. left pre-treatment traces vs right post-treatment traces), the spike activity can be quantified using software developed by D. Hochman that measures 1) Spike height, 2) Spike frequencty, and 3) Interspike intervals. Note that electrical stimulation-evoked ADs can be simultaneously generated and recorded at a sit distant to the bicuculline focus without interference.

### Example 6: Analysis of NPT 2024 (Bumetanide Morpholino Amide; n=4)

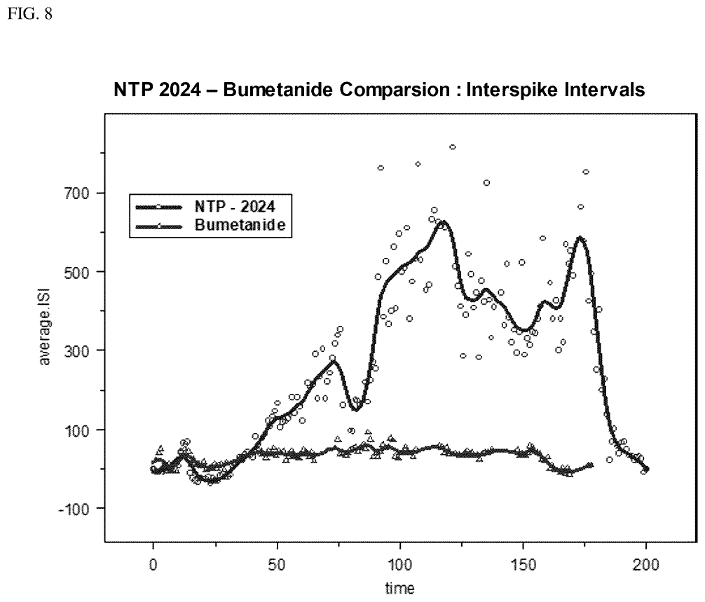

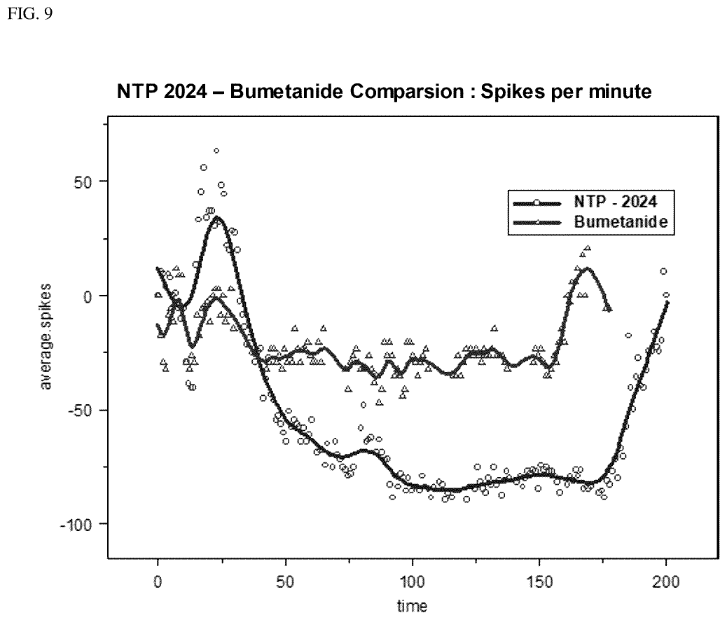

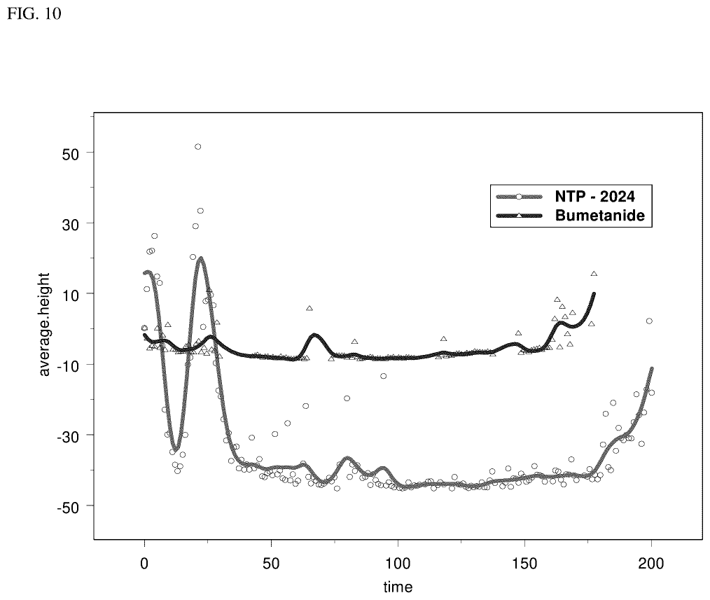

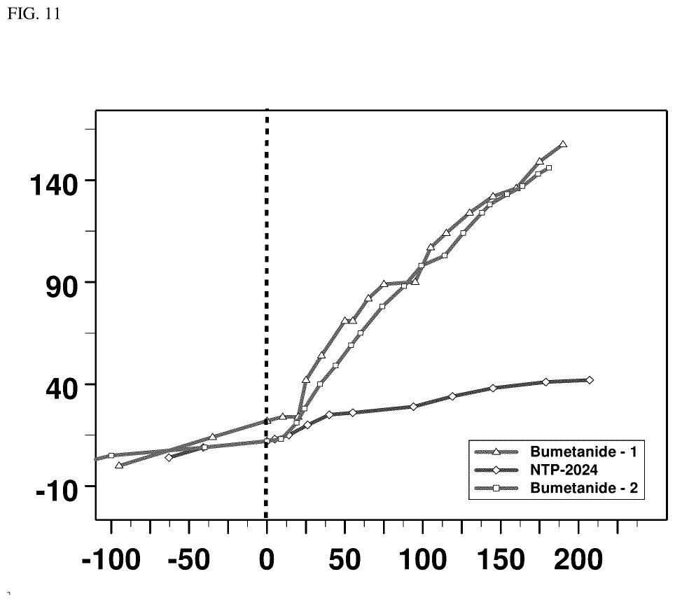

With reference to FIGS. 8-11, the results of comparative testing between the bumetanide morpholino amide derivative and the bumetanide parent compound demonstrates a dramatic increase in interspike intervals (FIG. 8), a dramatic decrease in spikes per minute (FIG. 9), a dramatic decrease in the average spike height (FIG. 10), and the comparative effect of NPT 2024 and bumetanide on urine production over time (FIG. 11).

### Example 7: The Antiseizure Effects of Oral and Intravenous Administration of Bumetanide Dibenzylamide (NPT 2042) in Nonhuman Primates

The purpose of the study was to assess the antiseizure effects of bumetanide dibenzylamide (NPT 2042) and as compared to bumetanide parent compound in nonhuman primates (NHP). As described hereinabiove, in these animals, an acute seizure focus was created on the neocortical surface with the GABAa antagonist bicuculline, to generate epileptiform “bicuculine spiking”. Single doses of either NPT 2042 or the reference control, bumetanide, were administered orally (PO) and intravenously (IV) after the seizure focus was created in these NHP. The bicuculline focus primate seizure model was used in this study because data previously acquired from primate seizure studies were qualitatively similar to what was observed when similar optical imaging studies were performed in patients with intractable epilepsy (Haglund and Hochman 2005). Specifically, the morphology and electrophysiological activity recorded in electroencephalogram (EEG) measurements was similar between NHP and humans.

Bicuculline blocks the inhibitory action of GABA receptors and elicits convulsions that are thought to be similar to epileptic seizures. Thus, bicuculline has been used in the laboratory to study and test the anticonvulsant effects of putative antiseizure treatments for decades (Schwartzkroin and Prince 1980). During the same experiments, the effects of NPT 2042 and bumetanide on diuresis were studied.

The results from the experiments indicate that both NPT 2042 and bumetanide elicited a near-complete blockade of the bicuculline spiking in the NHP seizure model following a 2 mg/kg intravenous dose administration. Oral doses of 10 mg/kg NPT 2042 and bumetanide both reduced spike height and frequency of bicuculline spiking. These studies demonstrate the ability of NPT 2042 and bumetanide to similarly suppress bicuculline spiking in a NHP seizure model.

The purpose of the study was to assess the antiseizure effects of bumetanide dibenzylamide (NPT 2042) in comparison to bumetanide in nonhuman primates. The results from this study confirm NPT 2042 as candidate to advance for clinical development as an adjunct antiseizure therapy in patients with medically intractable epilepsy. The test groups for intravenous and oral administration routes are shown in Table Ex 7-1 and Ex 7-2, respectively.

TABLE Ex 7-1
Test groups and sample size: intravenous route

| | | Test article dosage | Number of |
|---|---|---|---|
| Group | Treatment | (mg/kg/dose) | animals |
| 1 | IV bumetanide | 2 mg/kg | 1 |
| 2 | IV NPT 2042 | mol-Eq to 2 mg/kg | 1 |
| | | bumetanide | |

IV = intravenous.

TABLE Ex 7-2
Test groups and sample size: oral route

| | | Test article dosage | Number of |
|---|---|---|---|
| Group | Treatment | (mg/kg/dose) | animals |
| 1 | PO bumetanide | 10 mg/kg | 1 |
| 2 | PO NPT 2042 | mol-Eq to 10 mg/kg | 1 |
| | | bumetanide | |

PO = oral.

Bicuculline blocks the inhibitory action of GABA receptors and elicits convulsions that are thought to be similar to epileptic seizures. Thus, bicuculline has been used in the laboratory to study and test the anticonvulsant effects of putative antiseizure treatments for decades (Schwartzkroin and Prince 1980). Data acquired from primate seizure studies were qualitatively similar to what was observed from similar optical imaging studies in human patients in that the morphology of the electrophysiological activity recorded in EEG traces was similar between the NHP and humans (Haglund and Hochman, 2005).

Description of acute bicuculline seizure focus model: An acute “seizure focus” was created on the cortical surface. Three surface electrodes were place so that the motor electrode was overlying hand motor cortex, and another overlying hand sensory cortex. The middle electrode was used as a reference for differential recordings. The recordings electrodes could also be used to stimulate the cortex by passing current between a pair of electrodes. An image showing the placement of the surface electrodes is provided below is shown in FIG. 4

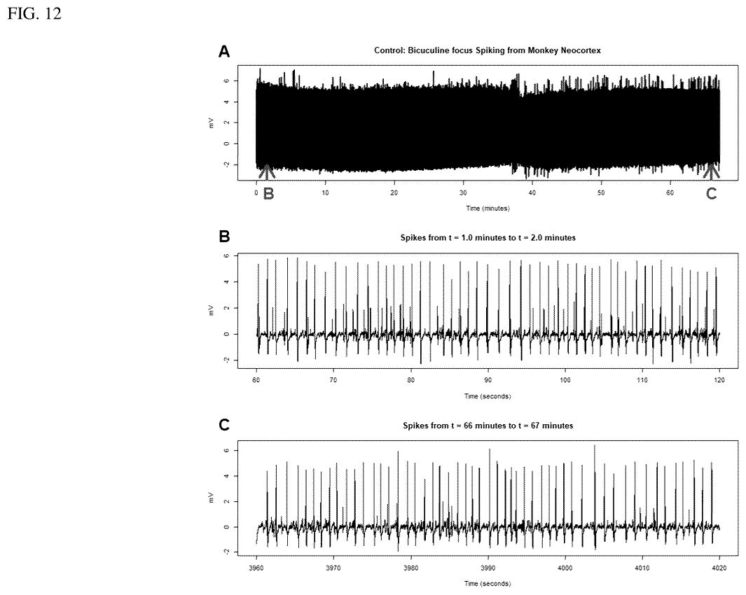

An acute “seizure focus” was created in the primate by placing a bicuculline-soaked pledget on the surface of the neocortex overlying arm/hand motor cortex for approximately 20 to 40 minutes until steady spiking activity (FIG. 12, panel A below) was observed. The pledget was then removed from the cortex, and the spiking would continue to persist for at least 4 to 6 hours until the experiment ended. The effects of various treaments on the biculline-evoked spiking could then be studied by administering treatments shortly after a consistent pattern of spiking had been elicited.

The A panel in FIG. 12 shows a continuous 70-minute segment of a trace recorded by a surface EEG electrode overlying the cortical area in which the bicuculline focus had been generated. Panels B and C show the first and last 60 seconds of the trace shown in A (at the times indicated by the red arrows in A), plotted at a faster time course so that the individual spikes can be seen. Importantly, no spontaneous significant changes in spike magnitude or frequency of spiking occur in this model.

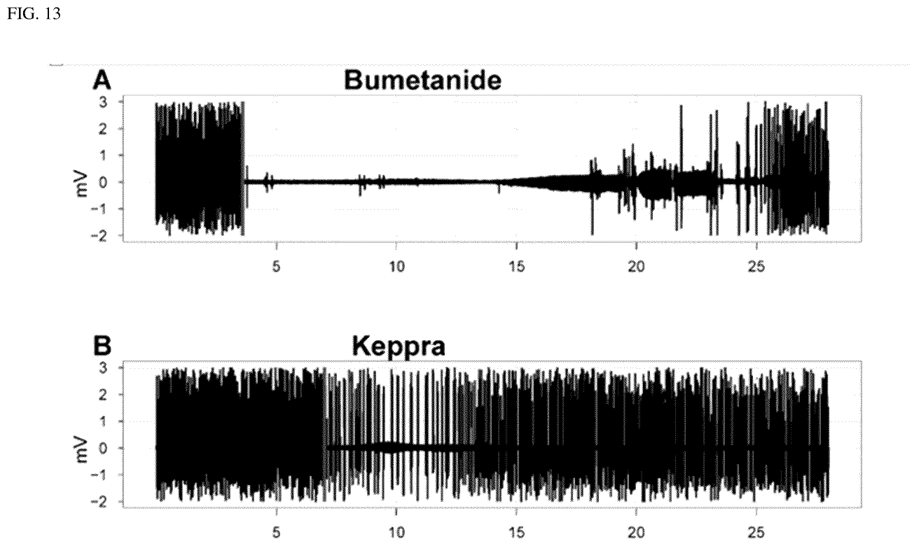

Comparison of the effects of bumetanide on bicuculline spiking with a standard of care treatment levetiracetam (Keppra®) is shown in FIG. 13. The tracing below demonstrates that bumetanide and Keppra both suppress bicuculline spiking. It is noteworthy that Keppra has a clear (though brief) effect on bicuculline spiking in this model, but it famously has no effect in any of the standard animal models used to screen for anticonvulsant activity (Loscher and Honack 1993).

### Experimental Design: Antiseizure Experiment

Macaque NHPs (Macaca nemestrina, weight 2 to 3 kg) were used in this experiment. Details on the treatment and surgical preparation of macaques for cortical AD stimulation and EEG recording have been described (Haglund et al. 1993; Haglund and Hochman 2007; Tolner et al. 2011) and briefly described below.

Animals were artificially ventilated on 100% oxygen via intubation (Matrix VBS anesthesia machine and Hallowell EMC model 2002 ventilator) and kept under pentobarbital anesthesia (1 to 2 mg/kg/hr). Oxygen saturation was measured from the tongue using a tongue-sensor (Nellcor Pulse Oximeter) and was continuously monitored to maintain a constant 98% to 100% saturation throughout the experiments.

Fluid balance was controlled by monitoring intravenous fluid intake every 15 minutes and fluid output (via a Foley catheter) every 30 minutes. Heart rate, blood pressure (Critikon Dinamap blood pressure monitor, model 8100), end-tidal partial pressure of carbon dioxide (pCO₂), respiration and O₂ levels (Ohmeda 5250 RGM anesthesia monitor; Nellcor NBP-40 for oxygen saturation [SpO₂]) were monitored in line with the EEG throughout experiments.

After craniectomy (25 mm in diameter), the dura was peeled back and surface EEG recordings were made using custom-built strip electrodes (2.5 mm electrode diameter; 1 cm interelectrode distance) placed over the sensorimotor cortex, with one electrode overlying the hand motor cortex, one over sensory cortex and a reference electrode attached to the skull at the mastoid process or to a third surface electrode in between the motor and sensory electrodes.

An acute bicuculline focus was generated in the motor cortex by 0.5 mm² pledgets of gelfoam (Codman & Shurtleff, Randolph, MA, USA) soaked in 100 μM bicuculline (Sigma-Aldrich, St. Louis, MO, USA). Pledgets were placed on the cortex for five minutes and refreshed for five minutes every hour. The bicuculline-evoked epileptiform spike activity stabilized after about 1 hour, after which data acquisition started. EEG-signals from motor and sensory cortex electrodes were continuously recorded (1000 Hz sampling, no filters; Axon Instrument's Digidata 1440A system).

Test products were administered once stable bicuculline spiking was observed in the EEG tracings were obtained for each NHP

Data were analyzed as described in a published reference (Haglund and Hochman, 2005) with custom-designed software using the R programming language. The bicuculline-induced spike activity was quantified from the motor cortex recording. The effect of test products on bicuculline-induced spikes was analyzed by comparing the peak response after a treatment was administered to the mean value during the 5 minutes control period before the test product administration (baseline). In the bicuculline experiments, recovery following test product administration was followed for a period of 20 to 35 minutes after return to pretreatment spike activity. For technical reasons and practical limitations on the staff's time, some recordings had to be terminated before the recovery phase was complete.

An example of EEG traces recorded with bicuculline spiking is shown in FIG. 12.

Test products: The investigational product is NPT 2042 and bumetanide is the reference product or active control. The product descriptions for NPT 2042 and bumetanide are listed in Table Ex 7-3 and Table Ex 7-4, respectively.

TABLE Ex 7-3
NPT 2042 product description

| Compound Code | NPT 2042 (bumetanide dibenzylamide) |
|---|---|
| CAS number | 885051-33-2 |
| Formula | C₃₁H₃₃N₃O₄S |
| Chemical name | 3-(Butylamino)-5-(dibenzylcarbamoyl)-2- |
| | phenoxybenzenesulfonimidic acid |
| Molecular weight | 543.68 g/mol |
| Estimated log(P) | 5.9 |

CAS = Chemical Abstracts Service.

TABLE Ex 7-4
Bumetanide product description

| Compound Code | Bumetanide |
|---|---|
| CAS number | 28395-03-1 |
| Formula | C₁₇H₂₀N₂O₅S |
| Chemical name | 3-(Aminosulfonyl)-5-(butylamino)-4- |
| | phenoxybenzoic acid |
| Molecular weight | 364.42 g/mol |
| Estimated log(P) | 2.61 |

CAS = Chemical Abstracts Service.

The investigational product, NPT 2042, was synthesized by Synexis, Inc. (Research Triangle Park, NC) and supplied to Duke University. NPT 2042 was stored at 2° C. to 8° C. The lot number of NPT 2042 was, 025DAP012 manufactured on Feb. 22, 2007.

A commercial source of the reference product, bumetanide was sourced by Duke University for the PO experiment.

For the IV experiment, a commercial source of the reference product, bumetanide was sourced by Synexis, Inc.

### Test Product Preparation

Each NHP was weighed prior to the study procedures and NPT2042 and bumetanide were administered at equimolar doses of 10 mg/kg PO and 2 mg/kg dose IV.

Each suspension of NPT 2042 and bumetanide was prepared for oral delivery (via gavage) by sonicating NPT 2042 or bumetanide in carboxymethyl cellulose (CMC) and water mixture. The suspensions were prepared immediately prior to use, kept at room temperature, and administered within 1 hour of preparation.

Each IV solution of NPT 2042 and bumetanide was prepared by dissolving NPT 2042 or bumetanide in 100% PEG-200. These solutions were supplied by Scynexis, Inc.

### Data Analysis

Data were analyzed as described previously with custom-designed software using the R programming language (Haglund and Hochman 2005).

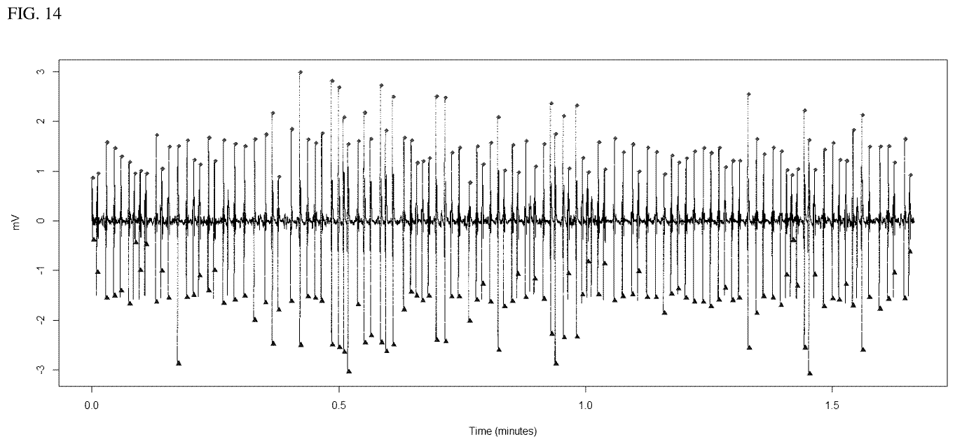

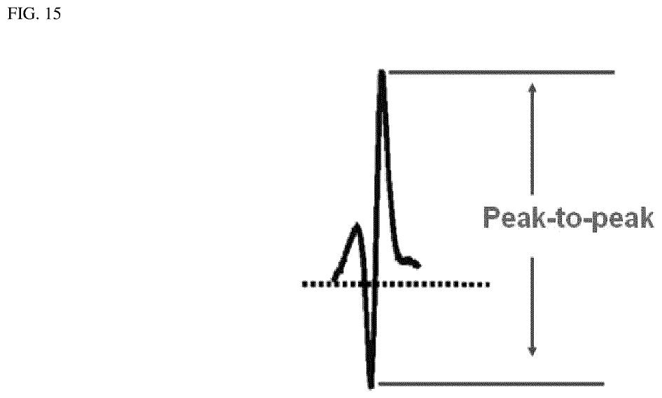

To better quantify the effect sizes from these recordings, an algorithm was applied to automatically detect the bottoms and tops of the bicuculine spikes, described herein. The algorithm is “brute force”, and works by moving a window across the data, whose width is just large enough to hold a single spike, and find the maxima and minima with the window, ignoring all changes that are less than two standard deviations above the background noise in the traces (Haglund and Hochman 2005). An example of a visual representation of the output from this computer analysis is shown below in FIG. 14. Here, the computer identified all the tops of spikes with upper (red) dots, and the corresponding bottoms of spikes with lower (blue) dots. The distance between the maxima and minima is the peak height is shown in FIG. 15.

From these data, the computer-generated calculation of the height of each spike, and the time intervals between each spike (interspike intervals, ISI). To reduce the likelihood of short-lived (1 or 2 minutes) random fluctuations biasing the analysis, the data (the spike sizes and ISIs) were smoothed using a moving-average window of 3 minutes. From these smoothed data, the following statistics were calculated for the oral administration of bumetanide and NPT 2042:1) spike sizes during a 10-minute pretreatment interval, and during a 10-minute interval around the time of the spikes identified to represent the maximum changes; 2) interspike intervals pretreatment and post-treatment, during the same time intervals that the spike sizes were obtained; and 3) time to 50% recovery—the time from the maximum change in spike size or ISI to 50% of the average value during pretreatment times. Because of the smoothing, the standard deviations over the 10-minute windows mentioned above were negligeable and therefore not reported.

### Results

### Intravenous Administration of Bumetanide and NPT 2042

In two experiments on two different animals, bumetanide (2 mg/kg) and NPT 2042 (mol-Eq to 2 mg/kg bumetanide) were administered via IV injection into an arm vein via the IV line that was used during the experiments to maintain the animals' health status.

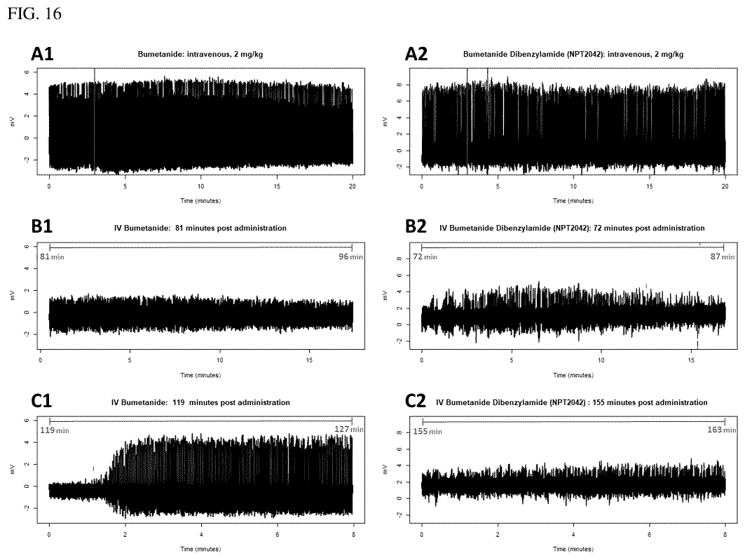

The EEG traces from this experiment are represented in FIG. 16. Row A traces in FIG. 16 show 20 continuous minutes of recordings, beginning 3 minutes prior to administration of either bumetanide (A1) or NPT 2042 (A2). The times of test product administration are shown by the red vertical bars in these traces (at t=3 minutes). Row B traces in FIG. 16 were selected from times when a profound, nearly complete blockade of the epileptiform activity was observed, starting at t=81 minutes postinjection for bumetanide (B1) and at t=72 minutes post injection for NPT 2042 (B2). Row C traces in FIG. 16 show the period of abrupt recovery to baseline pretreatment spiking conditions following bumetanide treatment beginning at about t=120 minutes postinjection (C1), and at the end of the experiment at t=155 minutes post-NPT 2042 injection (C2), where recovery to baseline had not yet been observed. For practical reasons, the experiment in which NPT 2042 was administered ended before time to recovery post-NPT 2042 administration could be determined.

### Oral Administration of Bumetanide and NPT 2042

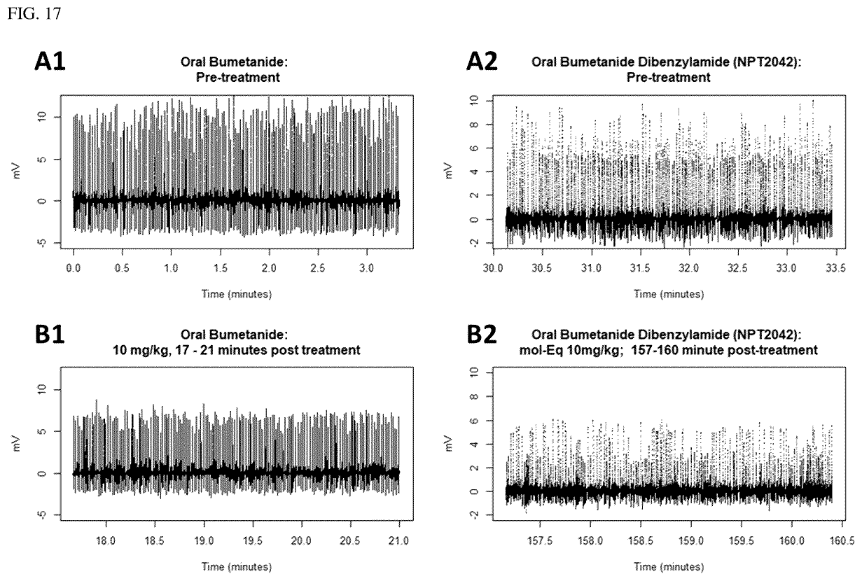

In two experiments in two different primates, bumetanide (10 mg/kg) and NPT 2042 (mol-Eq to 10 mg/kg bumetanide) were administered PO as an oral gavage once stable EEG tracings were obtained for each NHP. In contrast with the IV studies, where a nearly complete blockade of epileptiform activity was obvious and could be visually discerned from the raw data, the maximum changes elicited by oral administration were more subtle. FIG. 17 shows representative 3-minute pretreatment traces bumetanide (A1) and NPT 2042 (A2), and representative 3-minute traces during the maximum changes for bumetanide (B1) and NPT 2042 (B2). The traces in FIGS. A1 and A2 above represent a 3-minute pretreatment spike activity (baseline). The treatment effect is represented in the 3-minute interval in FIGS., B1 and B2. The results for PO bumetanide and NPT 2042 demonstrate that both compounds transiently mediate a reduction in size and frequency of spiking Table Ex 7-5. The experiment was not able to demonstrate quantitative differences between the compounds because of the small sample size and variability between animals.

TABLE Ex 7-5
Effect of PO bumetanide and NPT 2042
on bicuculline-generated spikes

| | Bumetanide PO | NPT 2042 PO | |
|---|---|---|---|
| Average % change in spike size | 23.7% | 35.2% | |
| Time to 50% recovery for spike sizes | 35 minutes | 160 | minutes |
| Average % change in ISI | 68.7% | 26% | |
| Time to 50% recovery of ISI | 163 minutes | 126 | nutes |

### Conclusions

There are limitations on what can be concluded from small sample sizes in these experiments. However, given the stability over time of the bicuculline spiking from the acute seizure focus created on the monkey cortex, and the near-complete blockade of that spiking shortly after intravenously administering bumetanide or NPT 2042, it seems likely that NPT 2042, similar to bumetanide reduces bicuculline-elicited epileptiform activity in the monkey. When quantified during PO experiments where there was a lesser effect from treatments, both bumetanide and NPT 2042 appeared to briefly reduce both spike size and frequency shortly following administration of the treatment, and both recovered to pretreatment values.

### Example 8: The Diuretic Effects of Oral Administration of Bumetanide Dibenzylamide (NPT 2042) in Nonhuman Primates

The purpose of this study was to assess the diuretic effects of bumetanide dibenzylamide (NPT 2042) and bumetanide in nonhuman primates (NHP). Urine output was measured from the same animals during the same experiments in which the effects of NPT 2042 and bumetanide on epileptiform EEG activity were studied (see Study No NPT RD 103). Urine from Foley catheterized Macaca nemestrina monkeys was collected into a graduated flask so that urine volumes could be measured at regular time intervals throughout the course of the experiments. Single doses of either NPT 2042 or the reference control, bumetanide, were administered orally (PO) to the anesthetized, catheterized primates.

These studies showed that a 10 mg/kg orally administered dose of bumetanide elicited a peak increase in urine production by at least 1500% of the control value in Macaque monkeys whereas equimolar doses of amide analog bumetanide dibenzylamide (NPT 2042) did not elicit a measurable diuretic response. It is important to mention that the seizure-suppressing effects of NPT 2042 were confirmed in the antiseizure arm of these studies (Example 7 hereinabove). In those experiments, equimolar oral doses of 10 mg/kg NPT 2042 and bumetanide both reduced spike height and frequency of bicuculline spiking.

Given the extremely large increases in diuresis elicited by bumetanide and the lack of any measurable increase by NPT 2042 in the nonhuman primates, this study demonstrates that NPT 2042 is much less diuretic than bumetanide, in this limited sample set.

### Study Purpose and Objectives

The purpose of the study was to assess the diuretic effects of bumetanide dibenzylamide (NPT 2042) in comparison to bumetanide in nonhuman primates. The results from this study were used to confirm NPT 2042 as drug candidate to advance for clinical development as an adjunct antiseizure therapy in patients with medically intractable epilepsy. A key attribute for drug candidate selection among the NKCC antagonist analog candidates is the decrease or reduction of diuresis so the drug can be tolerated as chronic treatment.

In this experiment two primates were evaluated, one received bumetanide and the other NPT 2042, via oral gavage, Table Ex 8-1.

TABLE Ex 8-1
Test groups and sample size

| | | Test article dosage | Number of |
|---|---|---|---|
| Group | Treatment | (mg/kg/dose) | animals |
| 1 | PO bumetanide | 10 mg/kg | 1 |
| 2 | PO NPT 2042 | mol-Eq to 10 mg/kg | 1 |
| | | bumetanide | |

PO = oral.

The data obtained for the assessment of urinary output following orally-administered bumetanide and NPT 2042 were obtained during the study evaluating the effects of these molecules on bicuculine-mediated spiking and cortical EEG recording. These primate studies were conducted in Dr. Hochman's laboratory while on faculty at Duke University Medical Center.

Details on the treatment and surgical preparation of macaques for EEG recording have been described (Haglund and Hochman 2007; Tolner et al. 2011).

### Experimental Design: Diuresis Experiment

For urinary output data catalogued in this technical report, briefly, animals were artificially ventilated on 100% oxygen via intubation (Matrix VBS anesthesia machine and Hallowell EMC model 2002 ventilator) and kept under pentobarbital anesthesia (1-2 mg/kg/h). Oxygen saturation was measured from the tongue using a tongue-sensor (Nellcor Pulse Oximeter) and was continuously monitored to maintain constant 98-100% saturation throughout the experiments.

Test and reference material was administered after baseline urine volume measurements were obtained (up to 100 minutes prior to test article administration). Urine production was measured after administration of 10 mg/kg oral bumetanide (n=3), and the molar equivalent (mol-Eq) to 10 mg/kg of oral bumetanide dibenzylamide administered orally (NPT 2042; n=1).

Fluid balance was controlled by monitoring intravenous fluid intake every 15 minutes and fluid output (via a Foley catheter) every 10 to 15 minutes until the animal returned to pretreatment levels of urine production. Heart rate, blood pressure (Critikon Dinamap blood pressure monitor, model 8100), end-tidal pCO₂, respiration and O₂ levels (Ohmeda 5250 RGM anesthesia monitor; Nellcor NBP-40 for SpO₂) were monitored in line with the EEG throughout experiments.

All urine samples were collected from the Foley catheter into a graduated flask and the volume was recorded for at least two time points prior to administration of the test article and reference material (to acquire an average baseline/pretreatment rate of urine production) for at least one hour prior to treatment administration, and every 15 minutes following treatment administration until a peak urine output and recovery was observed (for post-treatment rate of urine production).

### Test Products

The investigational product is NPT 2042 and bumetanide is the reference product or active control. The product descriptions for NPT 2042 and bumetanide are listed in Table Ex 8-2 and Ex 8-3, respectively.

TABLE Ex 8-2
Test product information

| Compound Code | NPT 2042 (Bumetanide Dibenzylamide) |
|---|---|
| CAS number | 885051-33-2 |
| Formula | C₃₁H₃₃N₃O₄S |
| Chemical name | 3-(butylamino)-5-(dibenzylcarbamoyl)-2- |
| | phenoxybenzenesulfonimidic acid |
| Molecular weight | 543.68 g/mol |
| Estimated log(P) | 5.9 |

CAS = Chemical Abstracts Service.

TABLE Ex 8-3
Bumetanide product description

| Compound Code | Bumetanide |
|---|---|
| CAS number | 28395-03-1 |
| Formula | C₁₇H₂₀N₂O₅S |
| Chemical name | 3-(Aminosulfonyl)-5-(butylamino)-4-phenoxybenzoic |
| | acid |
| Molecular weight | 364.42 g/mol |
| Estimated log(P) | 2.61 |

CAS = Chemical Abstracts Service.

The investigational product, NPT 2042, was synthesized by Synexis, Inc. (Research Triangle Park, NC) and supplied to Duke University. NPT 2042 was stored at 2° C. to 8° C. The lot number of NPT 2042 was 025DAP012 and was manufactured on 22 Feb. 2007.

A commercial source of the reference product, bumetanide was sourced by Duke University.

Test product preparation: Each NHP was weighed prior to the study procedures and NPT 2042 and bumetanide were administered at equimolar (mol-Eq) doses of 10 mg/kg PO.

Each suspension of NPT 2042 and bumetanide was prepared for oral delivery (via gavage) by sonicating NPT 2042 or bumetanide in carboxymethyl cellulose (CMC) and water mixture. The suspensions were prepared immediately prior to use, kept at room temperature, and administered within 1 hour of preparation.

Data analysis: Data were analyzed with custom-designed software using the R programming language.

### Results

The molar equivalent of 10 mg/kg bumetanide was used. It was administered by oral delivery, via oral gavage in anesthetized monkeys, as a water-cmc suspension. Tables Ex 8-4 and 8-5 show the average change in spike height and time to about 50% recovery after treatment.

TABLE Ex 8-4
Change Seizure activity after bumetanide and NPT 2042.

| | | NPT 2042 PO |
|---|---|---|
| | Bumetanide PO | (dibenzylamide) |
| Average % change in spike size | 23.7% | 35.2% |
| Time to 50% recovery for spike sizes | 35 minutes | 160 minutes |
| Average % change in ISI | 68.7% | 26% |
| Time to 50% recovery of ISI | 163 minutes | 126 minutes |

TABLE Ex 8-5
Change Seizure activity after morpholino amide and diethylamide.

| | Morpholino | |
|---|---|---|
| | Amide | Diethylamide |
| Average % change in spike size | 44% | 40% |
| Time to 50% recovery for spike sizes | 110 minutes | 59 minutes |
| Average % change in ISI | 37.5% | 65% |
| Time to 50% recovery of ISI | 180 minutes | 59 minutes |

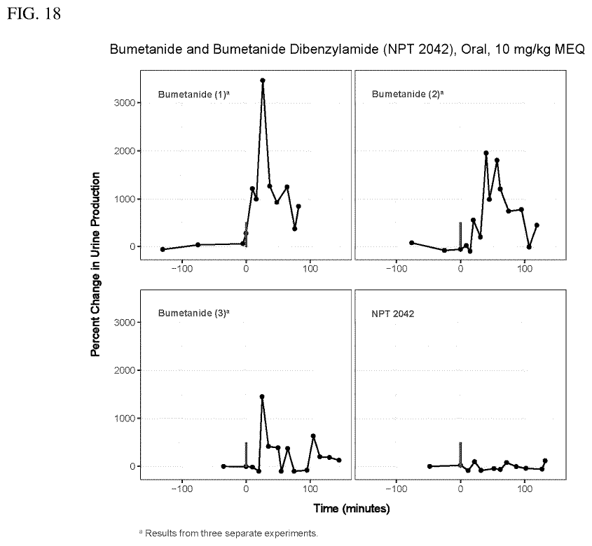

Each panel in FIG. 18 shows the percent change (with respect to baseline, pretreatment levels) in urine volume produced over time, for each individual monkey, after oral administration of bumetanide or NPT 2042. The diuretic response to bumetanide is represented in FIG. 18 in the graphs labeled Bumetanide (1) (2) and (3), and the remaining graph shows the diuretic response to bumetanide dibenzylamide (NPT 2042). The red line indicates the time the treatment was orally administered at t=0. Time is given in minutes on the x-axis, and the percent change in urine volume produced (by comparison to pretreatment urine production) is shown on the y-axis. The oral doses were 10 mg/kg of bumetanide and the molar equivalent of NPT 2042. Bumetanide elicited increases in urine volume production from over 1500% to 3000% of the baseline value, whereas the bumetanide amide analog, NPT 2042, elicited little or no increase in diuresis over baseline.

### Conclusions

These studies showed that 10 mg/kg orally administered bumetanide elicited a peak increase in urine production by at least 1500% in Macaque monkeys whereas the amide analog bumetanide dibenzylamide (NPT 2042) did not elicit any measurable diuretic response.

It is important to mention that the seizure-suppressing effects of NPT 2042 were confirmed in the antiseizure arm of these studies (see Study No NPT RD 103). In those experiments, equimolar oral doses of 10 mg/kg NPT 2042 and bumetanide both reduced spike height and frequency of bicuculline spiking.

Given the extremely large increases in diuresis elicited by bumetanide and the lack of any measurable increase by NPT 2042 in nonhuman primates, it seems likely that NPT 2042 is much less diuretic than bumetanide, even in view of the small sample size.

### Example 9: The Anxiolytic Effects of Intravenous Administration Bumetanide Dibenzylamide (NPT 2042) in Rats

The purpose of this study was to assess the central nervous system effects (anxiolytic effects) of bumetanide dibenzylamide (NPT 2042) in the rat fear-potentiated startle (FPS) model of conditioned anxiety (Lehmann et al. 2010; Krystal et al. 2012). The FPS model used in this study was the same model that previously evaluated the anxiolytic effects of two NKCC antagonists, bumetanide and furosemide (Krystal et al. 2012).

The FPS test includes two training sessions in which an intrinsically aversive foot shock is paired with a neutral cue light. In the test session, presentation of this cue light is subsequently used to elicit startle potentiation. The FPS procedure consists of five days of testing; baseline startle responses were collected during Days 1 and 2, light/shock pairings were delivered during Days 3 and 4, and the fear-potentiated startle was conducted on Day 5.

Animals were treated with either NPT 2042 (35 mg/kg), bumetanide (35 mg/kg), or vehicle (dimethyl sulfoxide [DMSO] only) on Day 5 through a cannulated jugular vein, 30 minutes prior to performing the FPS test. The doses selected in this study were determined from pilot studies performed by NeuroInvestigations, Inc. (Lethbridge, Canada) that determined minimal dose of bumetanide that elicited a measurable response in the FPS model (Data on file).

The startle amplitude was measured and compared between NPT 2042 versus vehicle and bumetanide versus vehicle.

The results indicate that NPT 2042 elicited an anxiolytic effect in the FPS model similar in magnitude to that elicited by equivalent (mg/kg) doses of bumetanide as determined by the reduction of the startle amplitude to a shock-conditioned stimulus.

### Study Purpose and Objectives

The purpose of this study was to evaluate the anxiolytic effects of bumetanide dibenzylamide (NPT 2042) in the rat FPS model. The results from this study confirm the suitability of NPT 2042 as drug candidate to advance into primate studies for further evaluation as a drug candidate as an adjunct antiseizure therapy in patients with medically intractable epilepsy.

In this study, Long-Evans male, adult (3 to 4 months old) rats were used to test for the anxiolytic effects of NPT 2042 in the FPS anxiety model (vehicle control/DMSO, n=51; bumetanide, n=14; NPT 2042, n=15). See Table Ex 9-1.

TABLE Ex 9-1
Test groups and sample size

| | | Test article | Number of |
|---|---|---|---|
| | | dosage | animals |
| Group | Treatment | (mg/kg/dose) | (sex) |
| 1 | Vehicle control | 0 | 51 (male) |
| 2 | Bumetanide: active control | 35 mg/kg | 14 (male) |
| 3 | NPT 2042: investigational drug | 35 mg/kg | 15 (male) |

For the purpose of screening bumetanide analogs, the rat model was the most reasonable model to measure a CNS response, and also feasible for testing and comparing a series of bumetanide analog candidates. Several standard rat epilepsy models were also considered but these models were highly labor-intensive, and/or required a large number of animals to yield statistically significant comparisons of the CNS effects of bumetanide to the bumetanide analogs and to each other. However, the rat associative anxiety models, particularly the fear-potentiated startle (FPS) model, showed extremely robust bumetanide-mediated NKCC-sensitive responses that varied accordingly to NKCC antagonist potencies and concentrations, yielded large and repeatable responses (so that far fewer animals needed to be used), and were far less labor-intensive than the rat seizure models (Krystal et al. 2012). It is noteworthy that a number of antiseizure drugs also have anxiolytic effects (Mula et al. 2007), and that rat anxiety models reliably predict therapeutic CNS responses in humans (Calabrese 2008) (see also Table 3 in (Krystal et al. 2012).

These rat studies were only intended to test CNS effects (in this case, reduced anxiety), and not anticipated diuretic effects of bumetanide since rats rapidly metabolizes and biotransforms bumetanide into inactive metabolites via oxidation of its N-butyl side chain before it can elicit diuresis through antagonism of renal NKCC2 (Schwartz 1981). Since the bumetanide analog candidates (including NPT 2042) have this same N-butyl side chain, they would be expected to be similarly vulnerable to such metabolism. The diuretic effects, therefore, have been evaluated in a study in primates and this is documented in report NPT RD 102 (NeuroPro Therapeutics Inc. 2022).

### Experimental Design: Fear-Potentiated Startle

A published FPS protocol (Lehmann et al. 2010) was followed and is described below.

Male adult (3 to 4 months old) Long-Evans rats, housed in the University of Lethbridge vivarium and under the direction of Janice Sutherland, PhD, were used for these studies. Rat housing consisted of Plexiglas cages with sawdust bedding shared with two or three rats. The colony room was temperature controlled (20° C. to 21° C.) with a 12 hour light/12 hour dark cycle, beginning each day at 7:00 am. Food and water were provided ad libitum. Seventy-two hours prior to the experiment, rats were anaesthetized with isoflurane, and a cannula was implanted into the right external jugular vein of each rat for the purpose of administration of test articles. Rats were thereafter kept in independent cages, and the cannulas were flushed daily to ensure patency.

All behavioral testing was conducted during the light cycle (7:00 am to 7:00 pm). Testing occurred between the hours of 9:00 am and 3:00 pm. Different randomly selected rats were used for each group (i.e., no rat was retested in more than one group). All testing was done under ambient room light.

Animals were trained and tested in four identical stabilimeter devices (Med-Associates). Each rat was placed in a small Plexiglas cylinder. The floor of each stabilimeter consisted of four 6 mm-diameter stainless steel bars spaced 18 mm apart through which shock could be delivered. Cylinder movements result in displacement of an accelerometer where the resultant voltage is proportional to the velocity of the cage displacement. Startle amplitude was defined as the maximum accelerometer voltage that occurred during the first 0.25 seconds after the startle stimulus was delivered. The analog output of the accelerometer was amplified, digitized on a scale of 0 to 4096 units and stored on a microcomputer. Each stabilimeter was enclosed in a ventilated, light- and sound-attenuating box. All sound level measurements were made with a Precision Sound Level Meter. The noise of a ventilating fan attached to a sidewall of each wooden box produces an overall background noise level of 64 dB. The startle stimulus was a 50 millisecond burst of white noise (5 millisecond rise to decay time) generated by a white noise generator. The visual conditioned stimulus was the illumination of a light bulb adjacent to the white noise source. The unconditioned stimulus was a 0.6 mA foot shock with duration of 0.5 seconds, generated by four constant-current shockers located outside the chamber. The presentation and sequencing of all stimuli were controlled by computer. Fear-potentiated startle procedures consisted of 5 days of testing. On Days 1 and 2 baseline startle responses were collected, on Days 3 and 4 light/shock pairings were delivered, and on Day 5 testing for fear potentiated startle was conducted. Animals received treatment with test compound or vehicle on Day 5.

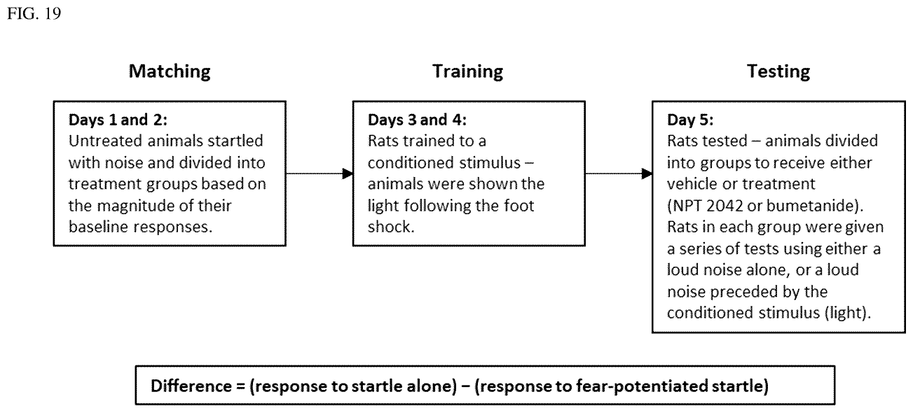

See FIG. 19 for an overview of the FPS model. Details describing the daily procedures are described in the sections that follow.

### Day 1 and Day 2: Matching

On Days 1 and 2, rats were placed individually into the Plexiglas cylinders and 3 min later presented with 30 startle stimuli at a 30 sec interstimulus interval. An intensity of 105 dB was used. The mean startle amplitude across the 30 startle stimuli on the second day was used to assign rats into treatment groups with similar means.

### Day 3 and Day 4: Training

On Days 3 and 4, rats were placed individually into the Plexiglas cylinders. During the first 3 min in the chamber the rats were allowed to acclimate, then 10 condition stimulus (CS) shock pairings were delivered. The shock was delivered during the last 0.5 seconds of the 3.7 second CSs at an average intertrial interval of 4 minutes (range, 3 to 5 minutes).

### Day 5: Testing

Animals received investigational drug (NPT 2042 [35 mg/kg] or bumetanide [35 mg/kg]) or vehicle (DMSO only) on Day 5 through a cannulated jugular vein 30 minutes prior to testing.

For testing, rats were placed in the same startle boxes where they were trained and after 3 min acclimation were presented with 18 startle-eliciting stimuli (all at 105 dB). These initial startle stimuli were used to again habituate the rats to the acoustic startle stimuli. Thirty seconds after the last of these stimuli, each animal received 60 startle stimuli with half of the stimuli presented alone (startle alone trials) and the other half presented 3.2 seconds after the onset of the 3.7 second CS (CS-startle trials). All startle stimuli were presented at a mean 30 second interstimulus interval, randomly varying between 20 and 40 seconds.

### Test Products

The investigational drug is NPT 2042, and the control products were bumetanide (active control) and DMSO (vehicle control). The product descriptions for NPT 2042 and bumetanide are listed in Table Ex 9-2 and Table Ex 9-3, respectively.

TABLE Ex 9-2
NPT 2042 product description

| Compound Code | NPT 2042 (bumetanide dibenzylamide) |
|---|---|
| CAS number | 885051-33-2 |
| Formula | C₃₁H₃₃N₃O₄S |
| Chemical name | 3-(Butylamino)-5-(dibenzylcarbamoyl)-2- |
| | phenoxybenzenesulfonimidic acid |
| Molecular weight | 543.68 g/mol |
| Estimated log(P) | 5.9 |

CAS = Chemical Abstracts Service.

TABLE Ex 9-3
Bumetanide product description

| Compound Code | Bumetanide |
|---|---|
| CAS number | 28395-03-1 |
| Formula | C₁₇H₂₀N₂O₅S |
| Chemical name | 3-(Aminosulfonyl)-5-(butylamino)-4- |
| | phenoxybenzoic acid |
| Molecular weight | 364.42 g/mol |
| Estimated log(P) | 2.61 |

CAS = Chemical Abstracts Service.

The investigational drug, NPT 2042, was synthesized by Synexis (Research Triangle Park, NC) and supplied to University of Lethbridge vivarium. NPT 2042 was stored at 2° C. to 8° C. NPT 2042 Lot number 009MPS023 was manufactured on Oct. 10, 2005.

Commercial sources of the active control product, bumetanide and vehicle control, DMSO, were procured by University of Lethbridge vivarium.

### Test Product Preparation

Dose solutions were prepared immediately prior to use, stored at room temperature prior to use, and administered within 3 hours of preparation.

Each rat was weighed prior to the study procedures and NPT 2042 and bumetanide were administered at doses of 35 mg/kg IV.

Each IV solution of NPT 2042 and bumetanide was prepared by dissolving NPT 2042 or bumetanide in 100% DMSO.

Data analysis: Data were entered into Excel spreadsheets and SPSS for data analysis. Independent sample t-tests are used to compare each treatment groups. The statistical programming language R was used to generate plots and perform statistical analysis. Welch two-sample t-tests (one sided) were used to compare NPT 2042 to vehicle, and bumetanide to vehicle.

### Results

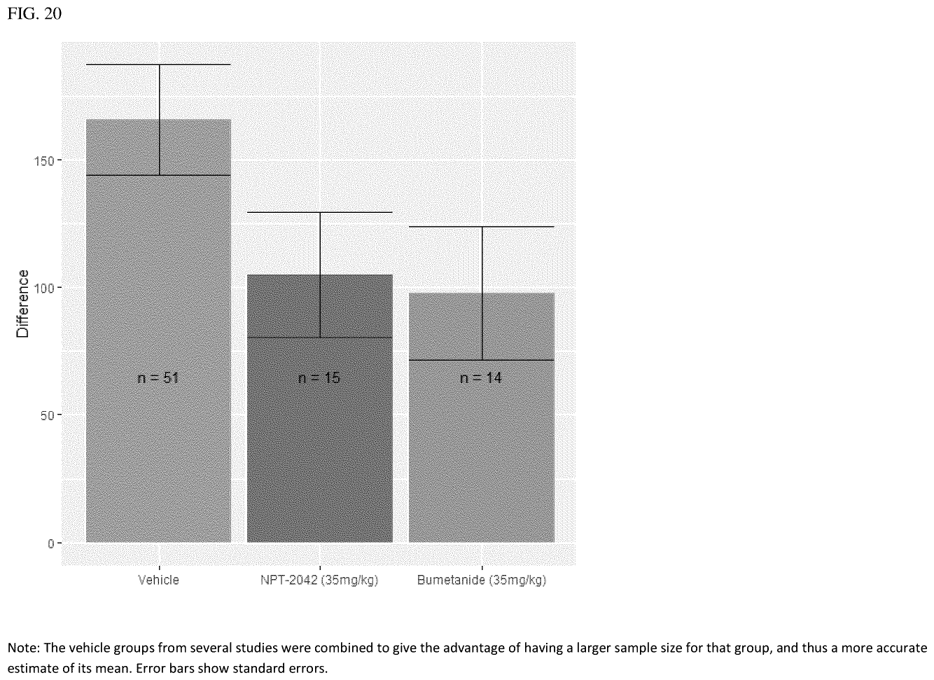

Both bumetanide and NPT 2042 had significantly less increase in startle amplitude with the shock-conditioned stimulus than rats treated with vehicle alone (vehicle mean=165.8 [standard error (SE)=21.6], p=0.02652; NPT 2042 mean=105 [SE=24.4], p=0.03512), bumetanide mean=97.6 (SE=26.3; p=0.02652). See, FIG. 20.

The 95% t-confidence intervals for the means are NPT 2042: [52.6, 157.4], bumetanide: [41.0, 154.2], Vehicle: [122.3, 209.3]

These data demonstrate that a 35 mg/kg dose of NPT 2042 has a similar CNS effect to a 35 mg/kg dose of bumetanide in the rat FPS model of conditioned anxiety.

No rats died in any of the test groups.

### Example 10

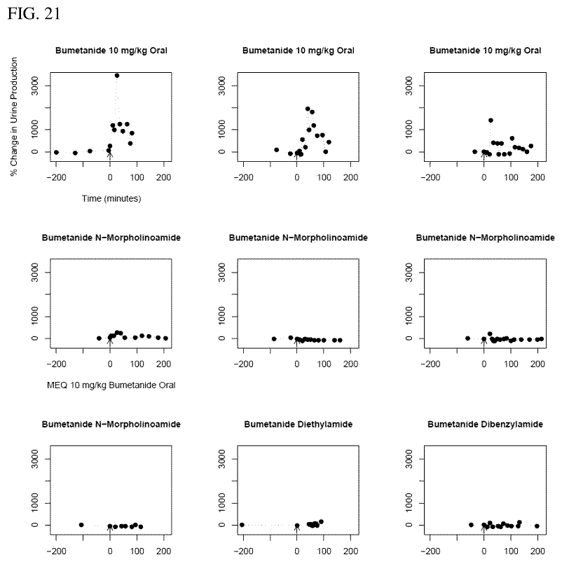

All three derivatives: bumetanide diethylamide, bumetanide N-morpholinoamide, and bumetanide dibenzylamide resulted in a lower percentage change in urine production over time compared to bumetanide. See, FIG. 21.

### Example 11

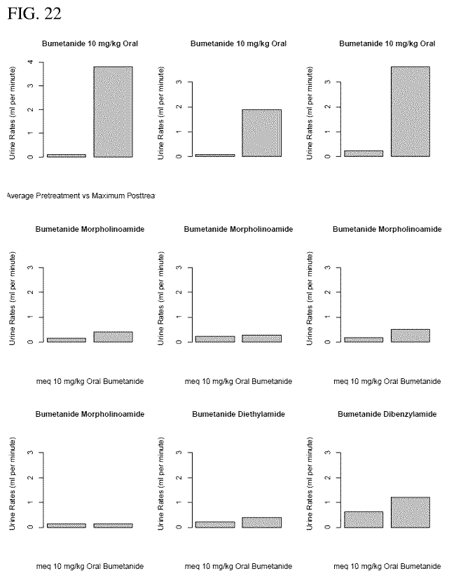

All three derivatives: bumetanide diethylamide, bumetanide N-morpholinoamide, and bumetanide dibenzylamide resulted in a lower rate of urine production over time compared to bumetanide. See, FIG. 22.

### Example 12

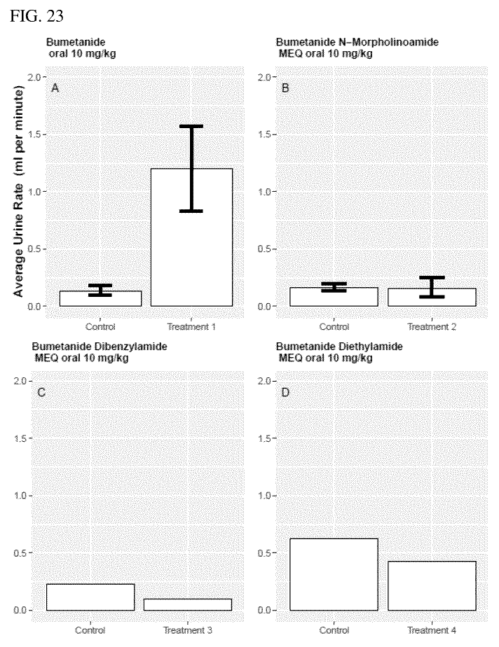

All three derivatives: bumetanide diethylamide, bumetanide N-morpholinoamide, and bumetanide dibenzylamide resulted in a lower average rate of urine production compared to bumetanide. See, FIG. 23.

### Example 13

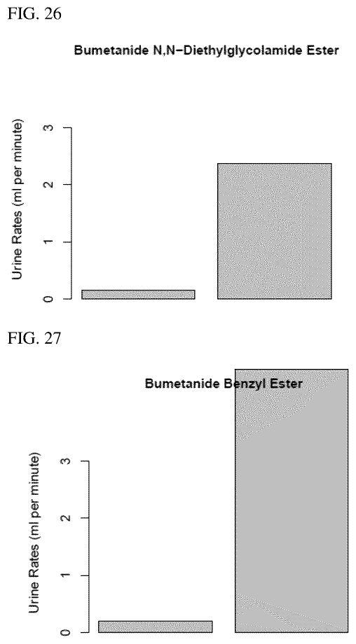

In direct contrast to the demonstrated results associated with the amide compounds, the ester prodrugs of bumetanide continued to present diuretic effects. A comparison may be made from FIG. 23 to FIGS. 24-27.

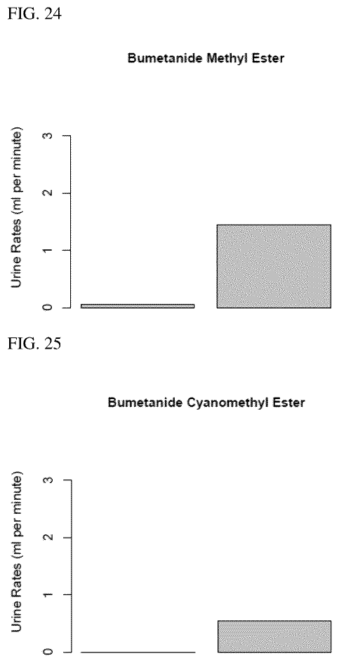

The average pretreatment urine rate (mL/min) is extremely low compared to the maximum post-treatment urine rate (mL/min) after treatment with esters including bumetanide methyl ester, bumetanide cyanomethyl ester, bumetanide N.N-diethylglycolamide ester, and bumetanide benzyl ester. See, FIGS. 24-27. All animals were administered the respective esters at a dose that was the molar equivalent to 2 mg/kg bumetanide. The administration was done intravenously. As shown, the diuretic effect of a bumetanide derivative is unpredictable.

Those skilled in the art to which the present disclosure pertains may make modifications resulting in other embodiments employing principles of the present disclosure without departing from its spirit or characteristics, particularly upon considering the foregoing teachings. Accordingly, the described embodiments are to be considered in all respects only as illustrative, and not restrictive, and the scope of the present disclosure is, therefore, indicated by the appended claims rather than by the foregoing description or drawings. Consequently, while the present disclosure has been described with reference to particular embodiments, modifications of structure, sequence, materials and the like apparent to those skilled in the art still fall within the scope as claimed.

### Example 14

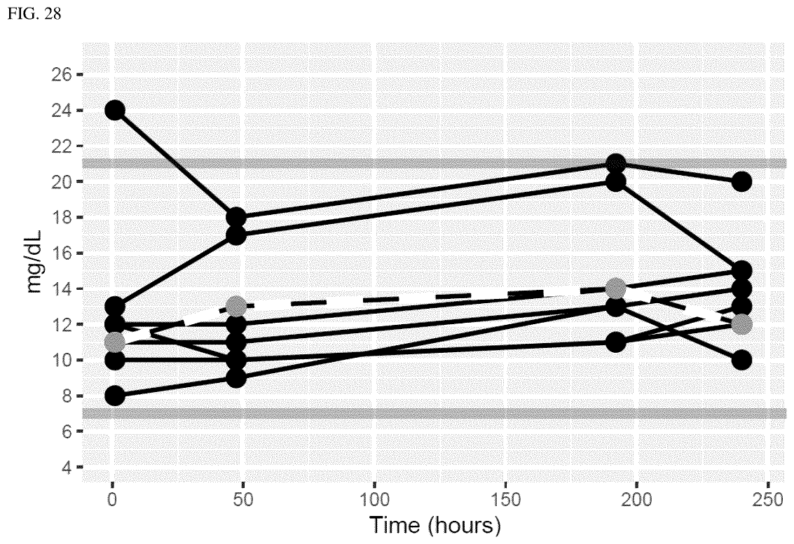

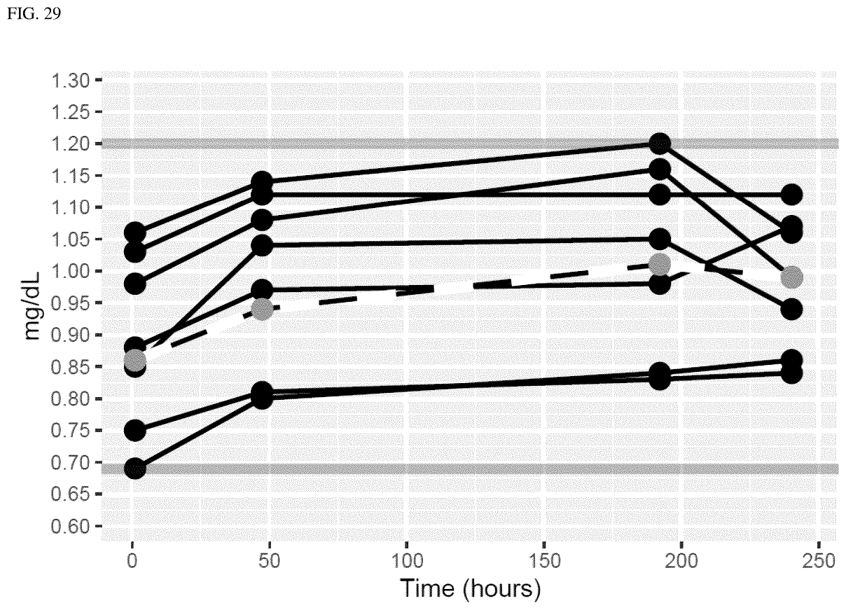

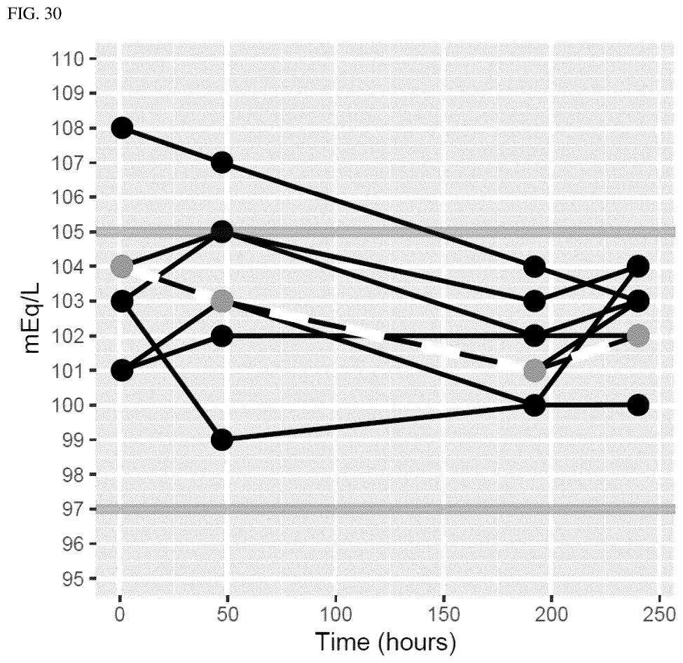

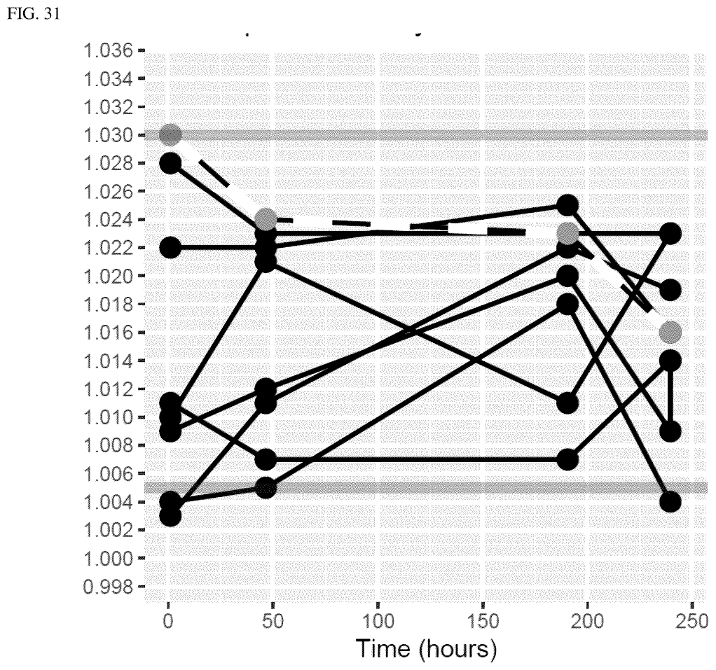

The purpose of this study was to assess the central nervous system effects (anxiolytic effects) of bumetanide dibenzylamide (NPT 2042) in humans to evaluate the safety and pharmacokinetics (PK) of single and repeated ascending doses of NPT 2042 in healthy adult subjects. The subjects were given eight capsules comprising 16 mg bumetanide dibenzylamide each every 24 hours. Pharmacokinetic (PK) blood samples were collected and analyzed pre-dosing to establish baseline levels. Analysis was also done on day1, day 3, day 4, day 5, day 6, day 7, and day 8 following dosing. The samples were analysed for BUN (blood urea nitrogen), creatinine, serum chloride, and urine specific gravity. Diuresis is indicated by an out of proportion increase in serum BUN compared to creatinine (BUN increase out of proportion to creatinine), a decrease in urine specific gravity, and an increase in serum chloride. As shown in FIGS. 28-29, there is no indication of diuresis. The data indicates that the bumetanide dibenzylamide is probably acting like a renal NKCC inhibitor. The grey horizontal lines in in FIGS. 28-29 are indicative of an upper and a lower range for accepted normal values. The control-subject (placebo) are indicated with white-and-black dashed lines and grey dots.

Those skilled in the art to which the present disclosure pertains may make modifications resulting in other embodiments employing principles of the present disclosure without departing from its spirit or characteristics, particularly upon considering the foregoing teachings.

Accordingly, the described embodiments are to be considered in all respects only as illustrative, and not restrictive, and the scope of the present disclosure is, therefore, indicated by the appended claims rather than by the foregoing description or drawings. Consequently, while the present disclosure has been described with reference to particular embodiments, modifications of structure, sequence, materials and the like apparent to those skilled in the art still fall within the scope as claimed.

What is claimed is:

1. A pharmaceutical composition comprising a Bumetanide prodrug or analog or derivative thereof, wherein the pharmaceutical composition has a therapeutic effect on seizure in a patient, wherein the therapeutic effect is a ratio of a measure of seizure suppression to a measure of diuretic effect on the patient.

2. The composition of any one of the claim 1, wherein the measure of seizure suppression is frequency of seizure.

3. The composition of any one of the claim 1, wherein the measure of seizure suppression is intensity of seizure.

4. The composition of any one of the claim 1, wherein the measure of seizure suppression is a change in amplitude of pharmacologically- or electrically-evoked seizure (epileptiform) activity as measured with EEG or other electrophysiological types of recordings.

5. The composition of claim 4, wherein the amplitude is decreased by about 50% to about 99% post treatment with the composition.

6. The composition of any one of the claim 5, wherein the measure of seizure suppression is a change in frequency of pharmacologically- or electrically-evoked seizure (epileptiform) activity as measured with EEG or other electrophysiological types of recordings.

7. The composition of any one of the claim 1, wherein the measure of diuretic effect is urine volume.

8. The composition of any one of the claim 1, wherein the measure of diuretic effect is urine ion concentration.

9. The composition of any one of the claim 1, wherein the therapeutic effect is based on at least one of an increase in interspike interval, seizure frequency, a change in blood plasma osmolarity, a change in urine production in a given time period, a change in blood ions over time, and a reduction in seizure spike height.

10. The composition of any one of the claim 9, wherein the blood ions are selected from sodium, chloride magnesium, or pH.

11. The composition of any one of the claim 1, wherein change is seizure frequency post treatment with the composition is at least a 50% reduction in frequency of seizure occurrence.

12. The composition of any one of the claim 1, wherein change is seizure frequency post treatment with the composition is a more than a 50% to a 100% reduction in the frequency of seizure occurrence.

13. The composition of any one of the claim 1, wherein, the measure of diuretic effect is a less than about two-fold increase in urine production over a twenty four hour period post treatment with the composition.

14. The composition of any one of the claim 1, wherein, the measure of diuretic effect is no increase in urine production over a twenty four hour period post treatment with the composition.

15. The composition of any one of the claim 1, wherein, the measure of diuretic effect is about a 0% to about a 100% increase in urine production over a twenty four hour period post treatment with the composition.

16. The composition of any one of the claim 1, wherein the therapeutic effect is determined based on an effective dose of the composition.

17. A pharmaceutical composition comprising a Bumetanide prodrug or analog or derivative thereof, wherein the pharmaceutical composition has a therapeutic effective on seizure blockade in a patient, wherein the therapeutic effect is determined as:
Therapeutic Effect=[seizure activity post-treatment]/[seizure activity pre-treatment]*[diuresis post-treatment]/[diuresis pre-treatment].

18. The composition of any one of the claim 17, wherein, the effective dose of the composition is a dosage required to completely block seizure activity.

19. The composition of any one of the claim 17, wherein, the effective dose of the composition is above the dosage required to completely block seizures.

20. The composition of any one of the claim 17, wherein, the effective dose of the composition is a dose that causes seizure suppression without causing the diuretic effect.

21. The composition of any one of the claim 17, wherein, the composition has a positive impact on neuron synchronous activity without a substantial impact on neuron excitability.

22. The composition of any one of the claim 17, wherein, the composition provides a therapeutic window of effect.

23. The composition of any one of the claim 17, wherein, the Bumetanide prodrug is Bumetanide Dibenzylamide.

24. A method for treating seizures in a patient comprising:
administering a pharmaceutical composition comprising a Bumetanide prodrug or analog or derivative thereof;
reducing seizure activity in the patient without increasing urine output of the patient.

25. The method of claim 24, wherein the pharmaceutical composition is administered orally.

26. The method of any one of the claim 24, wherein the pharmaceutical composition is administered once a day for a fixed number of consecutive days.

27. The method of any one of the claim 24, wherein an antiseizure effect of the pharmaceutical composition is mediated through its antagonism of NKCC1 on neurons or glial cells.

28. The method of any one of the claim 24, wherein a diuretic effects of the pharmaceutical composition is mediated through its antagonism of renal NKCC2.

29. The method of any one of the claim 24, wherein the pharmaceutical composition is administered once a day for a fixed number of consecutive days.

30. The method of any one of the claim 24, wherein the pharmaceutical composition is administered to treat epilepsy.
